SEQUENCE LISTING (8 sequences)

SEQ ID NO: 1 - DNA, 47 bp, Synthetic construct
  source 1..47: mol_type = other DNA
aggtgtcgtgacccgggatcatcagttactgtgttgactcactcagt

SEQ ID NO: 2 - DNA, 48 bp, Synthetic construct
  source 1..48: mol_type = other DNA
tatgatctagagtcgcggccacttcctacattgtgtcctctctcattg

SEQ ID NO: 3 - DNA, 11320 bp, Homo sapiens
  source 1..11320: mol_type = other DNA
atcagttactgtgttgactcactcagtgttgggatcactcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcagatcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtggccgcgactctagatcata

SEQ ID NO: 4 - DNA, 6026 bp, Synthetic construct
  source 1..6026: mol_type = other DNA
gttaactacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttctccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccccggttgataatcagaaaagccccaaaaacaggaagattgtataagcaaatattttactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagtggggcccgtgcaattgaagccggctggcgccaagcttctctgcaggattgaagcctgcttttttatactaacttgagcgaaatctggatcaccatgaaaacatttaacatttctcaacaagatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaattaaaagttttaccataccaagcttggctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagatccacgaattctgcagtcgacggtaccgcgggcccgggaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacccgggatccgccaccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgcccaagcttccgcggagccatggcttcccgccggcggtggcggcgcaggatgatggcacgctgcccatgtcttgtgcccaggagagcgggatggaccgtcaccctgcagcctgtgcttctgctaggatcaatgtgtaggcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatctggcgtaatagcgaagaggcccgcaccgatcgcccttcccaacagttgcgcagcctgaatggcgaatggcgcttcgcttggtaataaagcccgcttcggcgggctttttttt

SEQ ID NO: 5 - DNA, 2469 bp, Homo sapiens
  source 1..2469: mol_type = other DNA
ttattttagcaggaaaacatgtcccatgagacctatacacacacacattctgccttcagaattcagctgctgcattctgctgctgcttctcttattcagactgttggcatcatcaatacataacttttgattgaatcccaaggggaaaaaataactttggtagacagtggatacataacaaatgcatggattattctgggcattcctttttatttggtagagtgaaatttttggtgttgttgagaggataaaaaaggcatttaaaagtcaattttgaatccggattttctgctctgttaataaattcacatgaaagttacagaaagtattgttatgcttttgtactgaatagtttttgtgtttagaaggctttaaaagcaagtactatgtccactgtgctattctggtttggatattaatcagaacacagttgagcattgtttgaattcacagagcttgccatgctggaagcacaaccttatatgtagtgaccatggacagtcctattatgggaaaccaacttgagagagaaggcgggtcacttgcttgtgcgcaggtcctggaatttgaaatatccgggggcctctacagaatcctggcatgagggaacagctcaaaggccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggacacaatgtaggaagtcttttccacatggcagatgatttgggcagagcgatggagtccttagtatcagtcatgacagatgaagaaggagcagaataa

SEQ ID NO: 6 - DNA, 53 bp, Synthetic construct
  source 1..53: mol_type = other DNA
caaataaagatccacgaattcttattttagcaggaaaacatgtcccatgagac

SEQ ID NO: 7 - DNA, 40 bp, Synthetic construct
  source 1..40: mol_type = other DNA
aggtgtcgtgacccgggatcccctggcatgagggaacagc

SEQ ID NO: 8 - DNA, 53 bp, synthetic construct
  source 1..53: mol_type = other DNA
tatgatctagagtcgcggccgcttattctgctccttcttcatctgtcatgact

(19) United States
(12) Patent Application Publication
(10) Pub. No.: US 2025/0368705 A1
(43) Pub. Date: Dec. 4, 2025

(54) GENE-BASED MEDICINES AND CELLULAR THERAPY FOR DISEASE

(71) Applicants: CarryGenes Bioengineering, LLC, Savannah, GA (US); Artax Invest AB, Stockholm (SE)

(72) Inventors: Edward PERKINS, Savannah, GA (US); Amy L GREENE, Savannah, GA (US); Dominique BROCCOLI, Savannah, GA (US)

(21) Appl. No.: 19/226,097

(22) Filed: Jun. 2, 2025

Related U.S. Application Data
(60) Provisional application No. 63/655,597, filed on Jun. 4, 2024.
(60) Provisional application No. 63/722,598, filed on Nov. 19, 2024.

(51) Int. Cl. C07K 14/47; A61K 38/00; A61K 48/00; C12N 15/85

(52) U.S. Cl. CPC C07K 14/4708; A61K 48/005; C12N 15/85; A61K 38/00

(57) ABSTRACT

Herein disclosed are compositions comprising synthetic chromosomes and methods of their use to treat diseases and disorders (e.g., cancers, genetic and autoimmune diseases). Specifically described are methods of constructing synthetic chromosome compositions bearing one or multiple genes as well as regulatory sequences that control expression of the gene(s) such that, when these are expressed from the synthetic chromosome in an animal cell, at least one medicinal gene product is reliably, faithfully and indefinitely produced by the animal cells. As an example, the presently disclosed compositions and methods are used to bioengineer cells to enable them to express the entire dystrophin gene and additional regulatory nucleic acid sequences under tightly controlled conditions, allowing the present methods and compositions to be used as cellular medicines for treatment of diseases such as muscular dystrophies.

### STATEMENT REGARDING GOVERNMENT INTEREST

This invention was made with Government support under contracts D14PC00018 & D15PC00008 awarded by The United States Department of Defense Advanced Research Projects Agency (DARPA). The Government has certain rights in this invention.

### REFERENCE TO SEQUENCE LISTING SUBMITTED ELECTRONICALLY

A Sequence Listing (named “Artax101p2 SEQLIST2024-11-19.xml,” comprising 28,073 bytes and created on Jun. 1, 2025) is being submitted electronically via the U.S. Patent and Trademark Office's Patent Center electronic filing system in XML format, the content of which is herein incorporated by reference in its entirety.

### TECHNICAL FIELD

The present disclosure generally relates to advances in the field of synthetic chromosome bioengineering and cellular therapeutics for treatment of various diseases (e.g., cancers, genetic and autoimmune diseases). More particularly, provided herein are methods of constructing synthetic chromosome compositions comprising one or multiple genes that, when expressed in an animal cell, produce a therapeutic/medicinal gene product, and which chromosome compositions are readily portable into animal cells to achieve stable, transformative extra-genomic expression of medicinal agents to be used in cellular therapies.

### BACKGROUND

In the following discussion, certain articles and methods will be described for background and introductory purposes. Nothing contained herein is to be construed as an admission of prior art. Applicant expressly reserves the right to demonstrate, where appropriate, that the articles and methods referenced herein do not constitute prior art under the applicable statutory provisions.

Next-generation cell-based therapies for the treatment of disease will be buoyed by the development of portable, gene delivery systems that are capable of delivering large genetic inserts along with efficient safety switches. However, the development of current approaches to precision medicine applications and broad utility in a clinical setting has been hindered by impediments such as the risk of mutagenic events (e.g., unintentional or misplaced genomic integration of the introduced transgene), payload size limitations, and viral tropism. Fully functional, extragenomic, autonomous mammalian synthetic chromosomes (MACs) circumvent many of the limitations associated with plasmid and viral-based gene expression systems and provide an alternative means to introduce large segments of genomic DNA, sizeable cDNAs that exceed viral vector carrying capacity, developmentally regulated gene isoforms or splice variants, or multiple copies of two or more genes in fixed stoichiometry. The value and utility of a portable synthetic chromosome enabling the rational and tractable engineering of cells with multigene expression systems under control of one or more different expression controls, and/or large genomic fragments under native or designed regulatory control, or a combination thereof, and protected by effective safety switch mechanisms cannot be overstated. (Lindenbaum M, Perkins E, Csonka E, et al. Nucleic Acids Res. 2004, 32 (21): e172; Greene A L, Perkins E L. Methods Mol. Biol. 2011, 738:127-140; Greene A, Pascarelli K, Broccoli D, et al. Mol Ther Methods Clin Dev. 2019, 13:463-473; Stewart S, MacDonald N, Perkins E, et al. Gene Ther. 2002, 9 (11): 719-723; de Jong G, Telenius A, Vanderbyl S, Meitz A, Drayer J. Chromosome Res. 2001, 9 (6): 475-485).

Current gene therapies involving the use of viral-based vectors (e.g., AAV vectors) have limited value for multiple reasons: (1) viral-based expression vectors have limited nucleic acid carrying capacity (no more than approximately 5,000 bps) and are able to carry only fragments of very large genes and/or coding sequences over 5 kbps; (2) many people have pre-existing immunity against AAVs, making patients ineligible for treatment with AAV vector-based therapeutics; even FDA-approved viral delivery systems are known to have raised life-threatening immune responses in patients, leading to at least 11 deaths of patients treated with viral vector-based therapeutics; (3) the first dose of an AAV-based gene therapy agent can raise an immunogenic response which limits the prospect of repeated dosing; (4) therapeutic efficacy is affected by instability and loss of viral vectors over time (if not integrated into the host genome, viral vectors are episomal and are not stably conveyed by host cells indefinitely over many cell divisions, so their effects are short term and patients are prone to relapse; and (5) expression of the gene product has not been readily controllable. Preclinical work on potential AAV-based gene therapies requires a comprehensive analysis of safety, transgene expression in target and nontarget tissues to confirm the desired activity of the promoter only where intended, assessment of relevant functional measures of the transgene product, protein localization where appropriate, tissue-specific viral transduction, cellular impact, and vector tropism. (Asher, et al., Expert Opin. Biol. Ther., 2020, 20 (3): 263-274; Kaiser, J. Science, 2023 380 (6647): 778-779; Haseltine, W. A., “Gene Therapy Methods Explained,” Forbes, Apr. 16, 2024).

Thus, current gene therapies used for treatment of most diseases have provided neither a cure, nor even an enduring treatment, and a significant need for treatment of such diseases still remains.

In contrast, synthetic chromosome technology is aimed at genetic correction of a battery of diseases (e.g., cancers, genetic and autoimmune diseases) having biological mechanisms that are at least partially attributable to a lack or an excess of a gene product, and diseases and for which a corrective genetically encoded therapeutic agent can be supplied while minimizing risks, side effects and other negative consequences to a patient being treated. The presently described synthetic chromosome technology surpasses most if not all of the currently available genetic and cellular therapies by overcoming those negative outcomes and by providing a stable and long-term supply of a synthetic chromosome-encoded medicine under exquisitely controlled expression regulation.

The presently described therapeutic compositions encompass very tightly regulated mammalian synthetic chromosomes (mSynCs) and therapeutic cells carrying them, as well as methods of making and using them. These mSynCs have extraordinary benefits such as: (1) nearly unlimited nucleic acid carrying capacity; (2) both the expression of gene products from the synthetic chromosomes themselves as well as from the medicinal cells that carry them are very tightly regulatable and thus, the present system of gene therapy minimizes the risk of life-threatening immune responses in patients, and reduces the current requirement for co-treatment with immunosuppressive agents; (3) the therapeutic SynC compositions disclosed herein can be dosed multiple times; and (4) the mSynC compositions have long-term efficacy and can last indefinitely, possibly even for a lifetime.

The present disclosure provides a groundbreaking cell-based therapy designed to treat a multitude of diseases. The compositions and methods described herein are based on a non-viral system of cellular therapy that employs animal host cells bioengineered to stably carry an autonomously replicating synthetic chromosome that delivers medicinal cargo (e.g., the entire cDNA coding sequence of even the longest genes, entire genomic loci comprising introns and exons), and even multiple genes/genomic DNA sequences, and can further include regulatable promoter(s), genetic enhancer sequence(s), regulatory nucleic acid sequences, marker genes, safety switches and/or additional functional genetic sequences) to the host cells.

The innovative synthetic chromosome technology described herein solves a wide variety of problems associated with viral vector-based therapeutics and currently available cellular therapies and addresses the long-felt and unmet need of treating a wide variety of diseases having a genetic component. The therapeutic SynC compositions and methods disclosed herein provide a stable and effective treatment of the underlying genetic cause of many diseases, offering hope for improved outcomes and quality of life for individuals affected by a wide variety of diseases and disorders.

A nearly unlimited list of diseases, disorders and syndromes can be treated or ameliorated using the presently described cellular therapy employing mammalian synthetic chromosomes to deliver genetically encoded medicines that treat the underlying genetic cause(s) of disease. Examples of such diseases or disorders include, but are not limited to: aging-associated diseases, autoimmune diseases, endocrine diseases, growth disorders, eye diseases and disorders, hematological disorders, inflammations, injuries, intestinal diseases, infectious diseases, externally caused and/or environmental-related diseases, poisonings, metabolic disorders, sensory disorders (auditory, vision, olfaction), musculoskeletal diseases, neuromuscular diseases, connective tissue diseases, skin conditions, pre-cancers and cancers (e.g., carcinomas, sarcomas, leukemias, lymphomas, multiple myelomas, neoplasms, adenocarcinomas, germ cell tumors, blastomas, solid tumor cancers, neuroendocrine tumors, soft tissue cancers, neurological cancers, liposarcomas, bone cancers, muscle cancers, etc.). The presently described cellular therapy employing mammalian synthetic chromosomes can provide safer and longer-lasting patient outcomes than viral vector-based delivery systems.

One group of diseases that can be treated using the presently described compositions and methods are the muscular dystrophies; this group encompasses several neuromuscular and intrinsic muscle diseases causing progressive weakness and decreases in muscle mass in an affected individual over time, as well as compared to unaffected individuals. Duchenne Muscular Dystrophy (DMD) is the most common and severe form of inherited muscular dystrophies, occurring mostly in boys, although girls can be carriers and mildly affected. Mutations in the dystrophin gene interfere with the production of proteins needed to form healthy muscle, leading to progressive muscle atrophy, exhaustion of muscular regenerative capacity and concomitant muscle fiber degeneration, and overall muscular weakness. In individuals lacking or producing insufficient amounts of dystrophin, muscle cells become damaged and replaced with fat and fibrotic tissue.

The endogenous dystrophin gene is believed to be the largest gene in the human genome, spanning a genomic locus (Xp21.2-p21.1) of more than 2-million base pairs (2 Mbps) and encoding a large protein containing an N-terminal actin-binding domain and multiple spectrin repeats. Deletions, duplications, and point mutations at this gene locus may cause Duchenne muscular dystrophy (DMD), Becker muscular dystrophy (BMD), or cardiomyopathy. The encoded protein forms a component of the dystrophin-glycoprotein complex (DGC), which bridges the inner cytoskeleton and the extracellular matrix (ECM). Alternative promoter usage and alternative splicing result in numerous distinct transcript variants and protein isoforms for this gene.

The normal (wild type) dystrophin gene encodes an mRNA of about 16 kbps; thus, if the message encodes a single protein, it would be about 500 kD in size. However, the dystrophin gene has 79 exons and encodes a 427 kDa cytoskeletal protein. See (Asher, et al., Expert Opin. Biol. Ther., 2020, 20 (3): 263-274) for protein structure models of full-length protein and the mini- and micro-versions of dystrophin. For comparison, the murine dystrophin gene is 11,037 bps in length, the canine gene is 11,097 bps and human is 11,058 bps.

Approximately one in 3500 to 5000 males are affected by DMD. Symptoms usually first appear in childhood, between the ages of three and five years of age, and by age 12, many patients will need wheelchair assistance. Individuals with DMD often die in their twenties due to respiratory muscle weakness or cardiomyopathy. Signs and symptoms of DMD may include: progressive muscle weakness, muscle pain and stiffness, difficulty rising from a lying or sitting position (sometimes observed as a distinct pattern of movements known as “Gower's sign”), frequent falls, trouble running and jumping, waddling gait, walking on the toes, large calf muscles, delayed growth and/or learning disabilities.

Other types of muscular dystrophy may not surface until adulthood and may begin in different muscle groups. For example, Becker muscular dystrophy (BMD) shows signs and symptoms similar to those of DMD but tends to be milder and progress more slowly. Symptoms generally begin in the teens but might not occur until the mid-20s or later.

DMD and BMD have also been associated with diverse cognitive and behavioral comorbidities. Frameshift mutations in the DMD gene prevent the body-wide translation of dystrophin; besides a severe muscle phenotype, cognitive impairment and neuropsychiatric symptoms are often present in patients. Dystrophin protein 71 (Dp71) is the major DMD gene product expressed in the brain and mutations affecting its expression are associated with the DMD neuropsychiatric syndrome. As with dystrophin in muscle, Dp71 localizes to dystrophin-associated protein complexes in the brain. However, unlike in skeletal muscle; in the brain, Dp71 is alternatively spliced to produce many isoforms with differential subcellular localizations and diverse cellular functions, including neuronal differentiation, adhesion, cell division and excitatory synapse organization as well as nuclear functions such as nuclear scaffolding and DNA repair. (Naidoo, M and Anthony, K., 2019, Mol. Neurobiol. 57 (3): 1748-1767; doi: 10.1007/s12035-019-01845-w).

Genotype-phenotype studies have suggested that severity and risk of central defects in DMD patients increase with cumulative loss of different dystrophins produced in CNS from independent promoters of the DMD gene. Dp71 is the shortest isoform of dystrophin, and its contributions to cognitive, social, emotional, and behavioral dysfunctions as well as locomotor functions have been studied in a Dp71-null mouse model specifically lacking this short dystrophin. It was reported that that distal DMD gene mutations affecting Dp71 may contribute to the emergence of social and emotional problems that may relate to the autistic traits and executive dysfunctions reported in DMD. The present alterations in Dp71-null mice may possibly add to the subtle social behavior problems previously associated with the loss of the Dp427 dystrophin, in line with the current hypothesis that risk and severity of behavioral problems in patients increase with cumulative loss of several brain dystrophin isoforms. (Miranda, et al., Behav. Brain Funct., 2024, 20 (1): 21. doi: 10.1186/s12993-024-00246-x.).

Other types of muscular dystrophy may be defined by a specific feature or by where in the body symptoms begin, for example:

Limb-girdle muscular dystrophy (LGMD) usually begins in childhood or the teenage years and first affects hip and shoulder muscles, with the affected individual exhibiting difficulty lifting the front part of the foot, resulting in frequent stumbling.

Myotonic dystrophy is characterized by an inability to relax muscles following contractions, usually beginning in facial and neck muscles. In some cases, individuals having myotonic dystrophy may have long, thin faces, drooping eyelids, and a condition called “swan neck deformity” caused by damage in small muscles in the fingers and hands.

Facioscapulohumeral dystrophy (FSHD) typically begins as muscle weakness in the face, hip and shoulders during teenage years (although may appear in childhood or as late as age 50). Another symptom is sometimes observed when the subject's arms are raised and the shoulder blades look like wings.

Oculopharyngeal Muscular Dystrophy (OPMD) involves the pharyngeal muscles, resulting in swallowing disorders, and of the levator palpabrae superioris muscles, resulting in ptosis (Périé, S., Mol. Ther., 2014 January; 22 (1): 219-225).

Congenital muscular dystrophy affects boys and girls and is apparent at birth or before age 2. Some forms progress slowly and cause only mild disability, while others progress rapidly and cause severe impairment. At present, therapies for DMD are inadequate, largely focused on treatment of symptoms of the disease, such as physical therapy and the use of drugs such as glucocorticoids. Such drug treatments are merely palliative, often having adverse effects on the patient. (See Berry, S. E., Stem Cells Transl. Med., 2015 January; 4 (1): 91-98).

Skeletal muscle is the most abundant tissue of the body, and is an ideal target for cell therapy to slow the progression of congenital muscle diseases such as DMD or to regenerate injured tissue following trauma, as it is endowed with an excellent regenerative capacity due to its population of tissue-resident stem cells. Skeletal muscle consists predominantly of syncytial fibers with peripheral, post-mitotic myonuclei. Each individual muscle fiber and its associated muscle stem cells (MuSCs) are surrounded by a layer of extracellular matrix referred to as the basal lamina. Generally quiescent in postnatal life, at least a subset of undifferentiated MuSCs are capable of extensive self-renewal, allowing skeletal muscle to regenerate after repeated rounds of injury. The growth and repair of skeletal muscle fibers is mediated by a resident population of mononuclear myogenic precursors, the satellite cells, located between the sarcolemma and the basal lamina of the myofibers.

The progression of activated satellite cells toward myogenic differentiation is controlled by a family of transcription factors (myogenic regulatory factors; MRFs), including MyoD, Myf5, myogenin and MRF4. MuSCs are characterized by the expression of transcription factor Pax7, important for their self-renewal. In response to muscle tissue injury, the satellite cells become activated, enter the cell cycle and divide, giving rise to proliferating MyoD positive progenitors (myoblasts) before differentiating and fusing to repair damaged myofibers. Innovative in vitro strategies are guiding stem cell therapies for muscle repair towards the clinic. The syncytial nature of skeletal muscle uniquely permits the engraftment of stem/progenitor cells to contribute to new myonuclei and restore the expression of genes mutated in myopathies. (Judson, R. N. and Rossi, F. M. V., npj Regen. Med., 2020, 5 (10): 1-6; doi.org/10.1038/s41536-020-0094-3; Meregalli, et al., (2013) FEBS J., 280:4251-4262; Schüler, et al., Front. Cell. Dev. Biol., (2022) 10:1056523; doi: 10.3389/fcell.2022.1056523).

Thus, it follows that several muscular dystrophies can be ameliorated using the presently described cellular therapy employing a mammalian synthetic chromosome to deliver genetically encoded medicines that treat the underlying genetic cause(s) of this group of diseases and can provide safer and longer-lasting patient outcomes than currently available viral vector-based delivery systems. The present compositions and methods are based on a non-viral system of cellular therapy employing animal host cells bioengineered to stably carry an autonomously replicating synthetic chromosome for delivering, as the medicinal cargo, one or more extremely large expression cassettes comprising genetic sequences that can include, for example, the entire dystrophin locus, or the entire dystrophin gene comprising introns and exons, or even just a cDNA protein coding sequence, and optionally a second gene. Synthetic chromosomes having such cassette(s) can be ported into host cells which then can be administered to a patient having a muscular dystrophy needing genetic therapy. The synthetic chromosomes can further include regulatable promoter(s), genetic enhancer sequence(s), and regulatory sequences controlling the function thereof; marker genes; regulators that can turn the synthetic chromosome ON/OFF or can eliminate the chromosome-containing cells from the patient's tissue or body entirely; and/or additional functional genetic sequences to facilitate and/or modify expression of gene products in the host cells.

Additional background information about stem cell therapy for muscular dystrophy can also be found online at (//globalstemcells.com/treatment/muscular-dystrophy/).

Using the technology described herein, stem cells can be made to carry the presently described synthetic chromosome alongside their endogenous chromosomes and express a therapeutic gene, and these bioengineered medicinal cells are then administered to patients suffering from a disease to treat their disease reliably, faithfully and indefinitely. A DMD patient's autologous somatic cells can be reprogrammed to become induced pluripotent stem cells (iPSCs), bioengineered in vitro to carry the synthetic chromosome carrying the full-size dystrophin gene, and re-delivered to the patient as a cellular medicine to replace the missing or inadequate levels of the dystrophin protein.

In some instances, the present technology employing large-capacity mammalian synthetic chromosomes is used to deliver, express and stably convey in dividing cells even the largest and numerous genetic sequences in host cells, as well as to further deliver additional genes encoding gene products in consciously designed stoichiometric ratios and under highly-regulated expression control, genes encoding gene products to facilitate growth and survival of host cells, genes encoding gene products that facilitate identification and sorting of the host cells containing the synthetic chromosome, and/or genetic sequences that encode ON/OFF switches for regulation of the presence of the entire synthetic chromosome in host cells or even to eliminate the synthetic chromosome-carrying host cells themselves.

### BRIEF SUMMARY OF THE DISCLOSURE

The present disclosure provides synthetic chromosome compositions and methods for treating a wide variety of diseases, including autoimmune, endocrine, environmental, metabolic and genetic diseases, as well as cancers. Specifically, provided herein are methods of constructing synthetic chromosome compositions comprising one or multiple genes and transferring these compositions into animal host cells such that, when expressed in the cells, the synthetic chromosomes produce a therapeutic/medicinal gene product.

Accordingly, in some aspects, the present disclosure provides an autonomously replicating, stably inherited, non-integrating, non-native mammalian synthetic chromosome (mSynC) comprising: an rDNA-amplified centromere region, at least two telomeres, multiple copies of at least one type of unidirectional site-specific integration site, at least one of which site-specific integration sites comprises an irreversibly integrated genetic cassette greater than 5 kbp in size, wherein the integrated cassette comprises at least one therapeutic gene, a safety switch under tight expression control, and a marker allowing for identification of mSynC-bearing cells.

In some aspects, the mSynC further comprises at least one additional element selected from: a second therapeutic gene; a lineage-specific cellular differentiation gene and/or regulatory sequence; an enhancer of expression; a sequence encoding a cell-surface protein; a cellular growth factor; and a cytokine.

In some aspects, the therapeutic gene on the mSynC is present in multiple copies.

In some aspects, the mSynC used in cellular therapy treats or ameliorates diseases, disorders and syndromes such as aging-associated diseases, autoimmune diseases, endocrine diseases, growth disorders, eye diseases and disorders, hematological disorders, inflammations, injuries, intestinal diseases, infectious diseases, externally caused and/or environmental-related diseases, poisonings, metabolic disorders, sensory disorders (auditory, vision, olfaction), musculoskeletal diseases, neuromuscular diseases, connective tissue diseases, skin conditions, pre-cancers or cancers (e.g., carcinomas, sarcomas, leukemias, lymphomas, multiple myelomas, neoplasms, adenocarcinomas, germ cell tumors, blastomas, solid tumor cancers, neuroendocrine tumors, soft tissue cancers, neurological cancers, liposarcomas, bone cancers, and/or muscle cancers).

In some aspects, the therapeutic gene is involved in muscle function. In some embodiments, the therapeutic gene encodes a gene product that treats a muscular dystrophy. In some embodiments, the muscular dystrophy is selected from Duchenne Muscular Dystrophy (DMD), Limb-girdle Muscular Dystrophy (LGMD), myotonic dystrophy, Facioscapulohumeral Dystrophy (FSHD), Oculopharyngeal Muscular Dystrophy (OPMD) and congenital muscular dystrophy. In some embodiments, the therapeutic gene encodes a full-length dystrophin protein.

In some aspects, the mSynC comprises a second therapeutic gene selected from: another variant of the first therapeutic gene different from the first therapeutic gene, a second DMD gene that is a different variant than the first DMD therapeutic gene, DP71ab, utrophin, dysferlin, acetylgalactosaminyltransferase, GALGT2, PAX7, nestin, calpain 3, desmin, caveolin 3, and alpha-, beta-, delta- or gamma-sarcoglycan.

In some aspects, the mSynC further comprises at least one regulatory element that specifically regulates the second therapeutic gene.

In some aspects, multiple and different genes are present on the mSynC and, when inside host mammalian cells, express different gene products that treat a complex disease having multiple causes. In some embodiments, the complex disease has genetic components and/or is provoked by an external environmental stimulation or source. In some embodiments, the gene products are expressed in the host cells at different levels. In some embodiments, the gene products are components of a multi-protein complex. In some embodiments, regulatory control sequences such as promoters and/or enhancers control the amounts of gene products expressed to achieve a specific stoichiometry.

In some aspects, also provided herein is a method of controlling expression of a therapeutic gene in a host cell employing an mSynC.

In some aspects, also provided is a method of making a therapeutic cellular medicine by transferring a mammalian synthetic chromosome (mSynC) into a mammalian cell.

In some aspects, also provided is a method of cell-based therapy comprising: transferring, ex vivo, an mSynC into a mammalian cell, and administering the mSynC-carrying cells to a mammal in need of treatment. In some embodiments, the mammalian cell is a progenitor cell, a satellite cell, a smooth muscle cell, a cardiac muscle cell, a skeletal muscle cell, a myoblast, a myotube, a syncytium, or a sarcomere.

In some aspects, a method of cell-based therapy is provided, wherein the method comprises: isolating autologous somatic cells from a patient, reprogramming the patient-autologous cells to generate stem cells, transferring, ex vivo, an mSynC into the stem cells to generate transgenic patient-autologous stem cells, administering the transgenic patient-autologous stem cells carrying the mSynC to the patient.

A cell line accepting the mSynC should be capable of undergoing a number of cell divisions (i.e., not terminally differentiated).

In some embodiments, the method further comprises reprogramming the patient-autologous cells to generate cells selected from: induced pluripotent stem cells (iPSCs), mesenchymal stem cells (MSCs), MSCs derived from umbilical cord (ucMSCs), myoblasts, neural stem cells (NSCs), mesoangioblasts (MABs), and human iPSC-derived MAB-like cells (HIDEMs).

In some embodiments, reprogramming of cells (e.g., into progenitor cells) is performed before the mSynC is transferred into the cells. In some embodiments, reprogramming of cells (e.g., into progenitor cells) is performed after the mSync is transferred into the cells.

In some embodiments, somatic cells are used, and the somatic cells are satellite cells, myoblasts, vessel cells, myotubes, muscle cells, adipose cells, bone marrow cells, cells from synovium.

In some aspects, provided herein is a cellular medicine for treating a disease, comprising mammalian cells carrying an mSynC. In some embodiments, the cellular medicine treats the disease Muscular Dystrophy. In some embodiments, the muscular dystrophy is DMD.

In some aspects, provided herein is a host cell comprising the mSynC. In some embodiments, the host cell comprising the mSynC is a muscle cell. In some embodiments, the host cell comprising the mSynC is a stem cell.

In some aspects, provided herein is a cellular composition comprising a host cell carrying an mSynC and a pharmaceutically acceptable carrier.

### BRIEF DESCRIPTION OF THE FIGURES

FIG. 1: illustrates human synthetic chromosome (hSynC) system components and the bioengineering process;

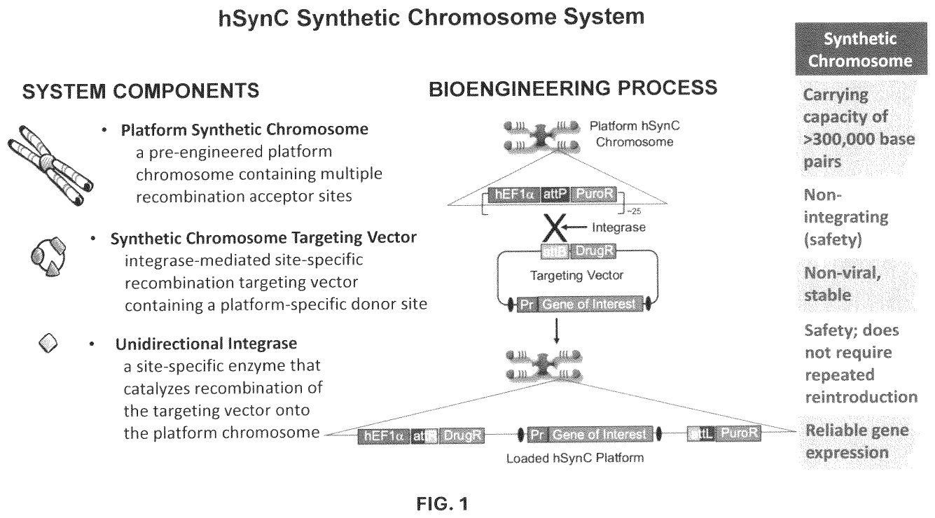

FIG. 2: describes examples of cell-based medicines and uses for the hSynC “circuit board”;

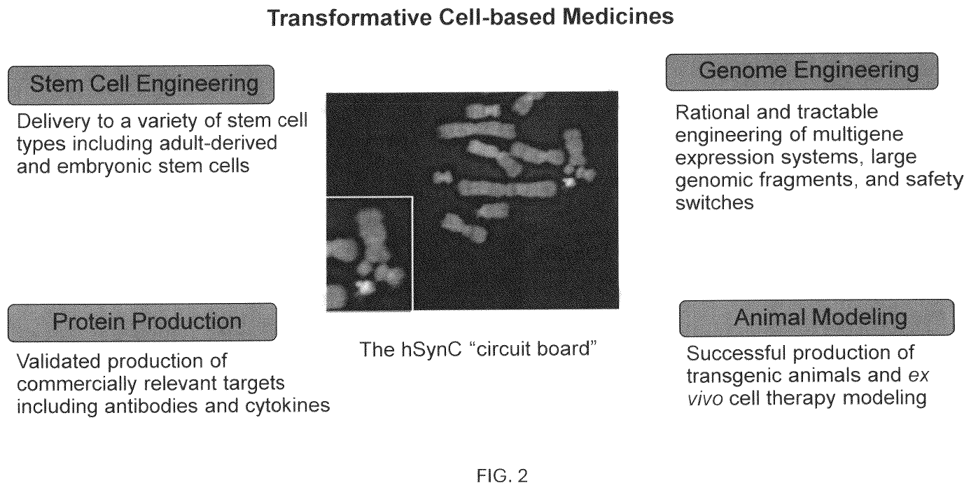

FIG. 3: presents the pEF1-Dp427m construct bearing a full-length dystrophin gene, and the pEF1-Dp71ab construct bearing the DP71ab fragment of the dystrophin gene;

FIG. 4: presents structural analyses of hSynC platform chromosome loadings with either the full-length dystrophin cDNA isoform Dp427m (plasmid pEF1aDMD-SYNp7) or the Dp71ab isoform variant (plasmid pEF1-Dp71ab). FIG. 4 (A) shows that 11 of 14 clones successfully loaded pEF1-Dp71ab onto the hSynC (˜78% success rate). FIG. 4 (B) shows that 15 of 18 clones successfully loaded pEF1-Dp427m onto the hSynC (˜83% success rate). 17 of 18 isolated cell clones loaded with the hSynC platform chromosome were found to contain the intact human DMD cDNA. FIG. 4 (C) indicates that genomic DNA from 17 of 18 isolated clones contained the intact human DMD cDNA including the first and last exons;

FIG. 5: shows RNA expression analysis of full-length dystrophin isoform Dp427m (pEF1aDMD-SYNp7) or isoform Dp71ab (pEF1aDp71ab) isolated from clonal cell lines;

FIG. 6: shows the hSynC targeting vector containing a proapoptotic cassette under the control of a proprietary doxycycline regulatable promoter;

FIG. 7: shows a Fluorescent in situ hybridization (FISH) image of human cells carrying the synthetic chromosome hSynC-Dp427m comprising the full-length DMD cDNA;

FIG. 8: shows that the DMD gene is expressed from the hSynC in impacted animals; and

FIG. 9: is a graphic illustration of the treatment of a patient having muscular dystrophy using the methods described herein.

### BRIEF DESCRIPTION OF THE SEQUENCES

SEQ ID NO: 1 represents the CGB0674 primer.

SEQ ID NO: 2 represents the CGB0635 primer.

SEQ ID NO: 3 represents the 11.3 Kb DMD (Dp427m) gene sequence cloned into the hSynC human synthetic chromosome.

SEQ ID NO: 4 represents the nucleic acid sequence of the vector construct SPB0487.

SEQ ID NO: 5 represents nucleic acid sequence of the small dystrophin Dp71 isoform.

SEQ ID NO: 6 represents the CGB683 primer.

SEQ ID NO: 7 represents the CGB684 primer.

SEQ ID NO: 8 represents the CGB685 primer.

### DETAILED DESCRIPTION

In this specification and the appended claims, numerous specific details are set forth to provide a more thorough understanding of the present invention. However, it will be apparent to one of ordinary skill in the art upon reading the specification that the present invention may be practiced without one or more of these specific details. In other instances, well-known features and procedures well known to those skilled in the art have not been described to avoid obscuring the invention.

As used herein, the singular forms “a,” “and,” and “the” include plural referents unless the context clearly indicates otherwise. Thus, for example, reference to “a protein” also includes more than one protein; reference to “a therapeutic agent” also includes more than one therapeutic agents, and so forth. Also, the use of “or” means “and/or” unless stated otherwise. Similarly, “comprise,” “comprises,” “comprising,” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.” As such, this statement is intended to serve as antecedent basis for use of such exclusive terminology as “solely,” “only” and the like in connection with the recitation of claim elements, or use of a “negative” limitation.

It is further noted that the claims may be drafted to exclude any optional element. The terms “optional” and “optionally” refer to a described event, circumstance, function, structure or other element may or may not occur, and that the description includes instances where the event or circumstance occurs and instances where the event, circumstance, function, structure or other element does not.

Before the present compositions, research tools and methods are described, it is to be understood that this invention is not limited to the specific methods, compositions, targets and uses described, as such may, of course, vary. It is also to be understood that both the foregoing general description, including the drawings, and the following detailed description are exemplary and explanatory only and are not restrictive of this disclosure. The section headings used herein are for organizational purposes only and not to be construed as limiting the subject matter described.

### Definitions

Unless specifically defined otherwise, the technical and scientific terms used in the description herein are intended to have the plain and ordinary meaning commonly understood by one of ordinary skill in the art. The following definitions are intended to aid the reader in understanding the present invention but are not intended to vary or otherwise limit the meaning of such terms unless specifically indicated.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Where a range of values is provided, it is understood that each intervening value between the upper and lower limit of that range and any other stated or intervening value in that stated range is encompassed within the invention. The upper and lower limits of these smaller ranges may independently be included in the smaller ranges, subject to any specifically excluded limit in the stated range. Where the stated range includes both of the limits, ranges excluding only one of those included limits are also included in the invention.

Unless defined otherwise, all technical and scientific terms used herein have the same meaning as commonly understood by one of ordinary skill in the art to which this invention belongs. All publications mentioned herein are incorporated herein by reference in their entirety for the purpose of describing and disclosing devices, formulations and methodologies which are described in the publication and which might be used in connection with the presently described invention.

The methods described herein may employ, unless otherwise indicated, conventional techniques and descriptions of molecular biology (including recombinant techniques), cell biology, biochemistry, and cellular engineering technology, all of which are within the skill of those who practice in the art. Conventional techniques include oligonucleotide synthesis, hybridization and ligation of oligonucleotides, transformation and transduction of cells, engineering of recombination systems, creation of transgenic animals and plants, and human gene therapy. Specific illustrations of suitable techniques can be had by reference to (but not limited to) some exemplary references herein, and equivalent conventional procedures. Some conventional techniques and descriptions can be found in standard laboratory manuals such as Genome Analysis: A Laboratory Manual Series (Vols. I-IV) (Green, et al., eds., 1999); Genetic Variation: A Laboratory Manual (Weiner, et al., eds., 2007); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Protein Methods (Bollag et al., John Wiley & Sons 1996); Nonviral Vectors for Gene Therapy (Wagner et al. eds., Academic Press 1999); Viral Vectors (Kaplift & Loewy, eds., Academic Press 1995); Immunology Methods Manual (Lefkovits ed., Academic Press 1997); Gene Therapy Techniques, Applications and Regulations From Laboratory to Clinic (Meager, ed., John Wiley & Sons 1999); M. Giacca, Gene Therapy (Springer 2010); Gene Therapy Protocols (LeDoux, ed., Springer 2008); Cell and Tissue Culture: Laboratory Procedures in Biotechnology (Doyle & Griffiths, eds., John Wiley & Sons 1998); Mammalian Chromosome Engineering—Methods and Protocols (G. Hadlaczky, ed., Humana Press 2011); Essential Stem Cell Methods, (Lanza and Klimanskaya, eds., Academic Press 2011); Stem Cell Therapies: Opportunities for Ensuring the Quality and Safety of Clinical Offerings: Summary of a Joint Workshop (Board on Health Sciences Policy, National Academies Press 2014); Essentials of Stem Cell Biology, Third Ed., (Lanza and Atala, eds., Academic Press 2013); and Handbook of Stem Cells, (Atala and Lanza, eds., Academic Press 2012), all of which are herein incorporated by reference in their entirety for all purposes.

Illustrative patents, published patent applications and non-patent publications that may be useful to aid description of the present compositions and methods, and possibly components or steps thereof, include but are not limited to the following:

U.S. Pat. No. 11,155,836 (Publication 2018/0010150, Ser. No. 15/548,236); U.S. Patent Publication 2022/0025404 (Ser. No. 17/482,307); U.S. Pat. No. 11,898,148 (Publication 2020/0157553, Ser. No. 16/092,828); U.S. Pat. No. 11,692,196 (Publication 2021/0403930, Ser. No. 16/092,837); U.S. patent application Ser. No. 18/217,601; U.S. Patent Publication 2019/0345259 (Ser. No. 16/092,841); U.S. Patent Publication US2020/0131530 (Ser. No. 16/494,252); U.S. Pat. No. 11,268,105 (Publication 2018/0171355, Ser. No. 15/844,014); U.S. Patent Publication 2022/0145322 (Ser. No. 17/582,609); U.S. Pat. No. 11,851,719 (Publication 2019/0071738, Ser. No. 16/120,638); U.S. patent Ser. No. 18/520,555); and PCT Application Nos. PCT/US2022/075512, PCT/US2022/075513, PCT/US2022/075520, PCT/US2022/075525; and PCT/US2022/075522; as well as U.S. Patent Provisional Application 63/655,597; each of which is incorporated by reference herein in its entirety for all purposes.

The present disclosure generally relates to advances in the field of synthetic chromosome bioengineering and cellular therapies for treatment of various diseases (e.g., cancers, genetic and autoimmune diseases). More particularly, provided herein are methods of constructing synthetic chromosome compositions comprising one or more very large genes, which chromosome compositions function as biological circuit boards and gene delivery systems that are readily portable into animal cells for stable, transformative extra-genomic expression of medicinal agents. The bioengineered synthetic chromosome compositions described herein can be used as gene delivery and expression vehicles to supply medicinal agents to higher-order eukaryotic (plant or animal) cells; furthermore, the synthetic chromosomes gene delivery and expression platform described herein can be used to design cellular therapies for treatment of diseases, and can be used in cell lines for in vitro/ex vivo production of commercially relevant proteins (e.g., cytokines or short chain variable fragments (scFvs)), nucleoproteins and protein-nucleic acid complexes (such as one or more proteins associated with DNA or RNA or both; e.g. signal recognition particles (SRPs), ribosomes, nucleosomes, viral nucleocapsids), and macromolecular complexes (e.g., antibodies). (Lindenbaum M, Perkins E, Csonka E, et al. Nucleic Acids Res. 2004, 32 (21): e172; Kennard M L, Goosney D L, Monteith D, et al. Biotechnol. Bioeng. 2009; 104 (3): 540-553).

As used herein, the following terms are intended to have the following meanings:

As used herein, “synthetic platform chromosome” means a synthetic chromosome construct (having multiple site-specific integration sites) ready to be engineered to bear one or more stably integrated expression cassettes; the synthetic platform chromosome refers to the construct before an expression cassette is added. The phrase “synthetic chromosome” refers to the synthetic chromosome after at least one expression cassette has been stably integrated.

A synthetic platform chromosome or a synthetic chromosome carrying at least one stably integrated expression cassette can be contained within a plant or animal host cell (in which case it functions as an autonomously replicating non-native bioengineered chromosome that autonomously replicates alongside the cell's endogenous chromosomes, and faithfully segregates to daughter cells during meiotic and/or mitotic cell divisions). Alternatively, a synthetic platform chromosome or synthetic chromosome carrying at least one stably integrated expression cassette can be isolated and not within the context of a plant or animal host cell.

Provided herein are therapeutic composition and methods that involve eukaryotic (most often mammalian) cells bearing a mammalian synthetic chromosome that autonomously replicates and is stably maintained over the course of at least 10 cell divisions. A mammalian synthetic chromosome (mSynC) (e.g., a human synthetic chromosome (hSynC)) as described herein is stably maintained over the course of at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 10,000, at least 20,000, at least 30,000, at least 40,000, or at least 50,000 cell divisions. It should also be noted that transgenic mammals bearing the presently disclosed mSynC have been demonstrated to be functionally intact from the single cell stage and onward to the adult animal, and thus, the mSynCs described herein have been stable over the course of millions of cell divisions. The mSynCs described herein are stable and express genes for a lifetime of the mammal.

In some embodiments, included on the mSynC are cellular growth, replication and survival facilitators, such as genes encoding growth factors (GFs), for facilitating culture of cells with or without the mSynC in vitro, cytokines, homing factors, as well as for facilitating growth, replication and survival of target cells or tissues in the body of a subject/patient (e.g., growth factors to induce muscle cell growth or tissue hypertrophy). Additional facilitators can be gene regulatory and/or gene expression control elements such as locus control regions, insulator elements, enhancers, constitutive or inducible (activatable or repressible) promoters.

In addition to the genetic and environmental diseases and disorders, the compositions and methods described herein may also treat infections of a subject by a viral, bacterial, fungal or protozoal pathogen. In some embodiments, the antigenic material is a virus, bacterium, fungus or protozoan itself. In some embodiments, a vaccine may be employed, potentially as a secondary therapeutic agent, wherein the virus, bacteria, fungi or protozoa is attenuated but live virus, bacteria, fungi, or protozoa. A live virus, bacteria, fungi, or protozoa is sufficiently attenuated to eliminate or reduce the occurrence of an infection when administered as a vaccine in conjunction with the compositions described herein.

At various places in the present specification, substituents of compounds of the present disclosure are disclosed in groups or in ranges. It is specifically intended that the present disclosure include each and every individual sub-combination of the members of such groups and ranges.

About: As used herein, the term “about” means+/−10% of the recited value.

Activity: As used herein, the term “activity” refers to the condition in which things are happening or being done. Compositions described herein may have activity and this activity may involve one or more biological events.

Adeno-associated virus: The term “adeno-associated virus” or “AAV” as used herein refers to members of the Dependoparvovirus genus comprising any particle, sequence, gene, protein, or component derived therefrom. The term “AAV particle” as used herein comprises a capsid and a polynucleotide. The AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

Administered in combination: As used herein, the term “administered in combination” or “combined administration” means that two or more agents (e.g., an mSynC and a small molecule drug) are administered to a subject at the same time or within an interval such that there may be an overlap of an effect of each agent on the patient and/or the subject is at some point in time simultaneously exposed to both. In some embodiments, they are administered within about 60, 30, 15, 10, 5, or 1 minutes of one another or within about 24 hours, 12 hours, 6 hours, 3 hours of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatus in administration. In some embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Among their many uses, synthetic chromosomes can be engineered to act as circuit boards for synthetic biology and construction of transgenic organisms (animals or plants) of agricultural importance. The presently described synthetic chromosomes provide the scalability (a predictable manner to construct complex genetic circuits) and orthogonality (the ability to alter engineering system components without influencing the performance of the components) necessary for the development of a directed, multi-trait approach to address climate change adaptation. Agriculturally important animals also can be bioengineered to express beneficial traits for survival in harsher conditions and/or a changing climate. Combining multiple identified traits on a portable synthetic chromosome that confer adaptative benefits in response to climate change stress would greatly accelerate the production of transgenic, climate acclimated animals bypassing the traditional, and time consuming, breeding strategies.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biologically active: As used herein, the phrase “biologically active” refers to a characteristic of any substance (e.g., a nucleic acid, a protein, a virus, a BAC based vector, a synthetic chromosome) that has activity in a biological system and/or organism. For instance, a substance that, when administered to an organism, has a biological effect on that organism, is considered to be biologically active. In particular embodiments, a polynucleotide of the present invention may be considered biologically active if even a portion of the polynucleotides is biologically active or mimics an activity considered biologically relevant.

Biological system: As used herein, the term “biological system” refers to a group of organs, tissues, cells, intracellular components, proteins, nucleic acids, molecules (including, but not limited to biomolecules) that function together to perform a certain biological task within cellular membranes, cellular compartments, cells, tissues, organs, organ systems, multicellular organisms, or any biological entity. In some embodiments, biological systems are cell signaling pathways comprising intracellular and/or extracellular cell signaling biomolecules. In some embodiments, biological systems comprise growth factor signaling events within the extracellular/cellular matrix and/or cellular niches.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pairs in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated. Perfect complementarity or 100% complementarity refers to the situation in which each nucleotide unit of one polynucleotide strand can form a hydrogen bond with a nucleotide unit of a second polynucleotide strand. Less than perfect complementarily refers to the situation in which some, but not all, nucleotide units of two strands can form hydrogen bonds with each other. For example, for two 20-mers, if only two base pairs on each strand can form hydrogen bonds with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Compounds and salts present in the compositions of the present disclosure can be prepared in combination with solvent or water molecules to form solvates and hydrates by routine methods. “Hydrate” refers to a combination of water with a solute, such as a drug or composition described herein, wherein the water retains its molecular state as water and is either absorbed, adsorbed or contained within a crystal lattice of the solute.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 96%, about 97%, about 98%, about 99% or 100% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide, a polynucleotide or polypeptide or may apply to a portion, region or feature thereof.

In one embodiment, conserved sequences are not contiguous. Those skilled in the art are able to appreciate how to achieve alignment when gaps in contiguous alignment are present between sequences, and to align corresponding residues notwithstanding insertions or deletions present.

In one embodiment, conserved sequences are not contiguous. Those skilled in the art are able to appreciate how to achieve alignment when gaps in contiguous alignment are present between sequences, and to align corresponding residues not withstanding insertions or deletions present.

Delivery: As used herein, “delivery” refers to the act or manner of delivering a compound (such as a delivery vector comprising a recombinantly engineered nucleic acid cassette bearing a gene to be inserted into an mSynC), substance, entity, moiety, cargo or payload to a target. Such target may be a nucleic acid, a vector, a cell, a tissue, an organ, an organism, or a system (whether biological or production).

Delivery Agent: As used herein, “delivery agent” refers to any agent such as a delivery vector, a nucleic acid, a bacterial or viral vector, or other chemical substance which facilitates, at least in part, the in vivo and/or in vitro delivery of a polynucleotide (e.g., a bioengineered mSynC) and/or one or more substances (including, but not limited to a compound and/or composition of the present invention, e.g., an mSynC) to targeted cells.

Destabilized: As used herein, the term “destable,” “destabilize,” or “destabilizing region” means a region or molecule that is less stable than a starting, reference, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance immunological detection, and the like. Detectable labels may include radioisotopes, fluorophores, chromophores, enzymes, dyes, metal ions, ligands such as biotin, avidin, streptavidin and haptens, quantum dots, and the like. Detectable labels may be located at any position in the entity with which they are attached, incorporated or associated. For example, when attached, incorporated in or associated with a peptide or protein, they may be within the amino acids, the peptides, or proteins, or located at the N- or C-termini.

Dosing regimen: As used herein, a “dosing regimen” is a schedule of administration or physician determined regimen of treatment, prophylaxis, or palliative care.

Effective Amount: As used herein, the term “effective amount” of an agent is that amount sufficient to effect beneficial or desired results, for example, upon single or multiple dose administration to a subject cell, in curing, alleviating, relieving or improving one or more symptoms of a disorder, clinical results, and, as such, an “effective amount” depends upon the context in which it is being applied. For example, in the context of administering an agent that treats Parkinson's Disease, an effective amount of an agent is, for example, an amount sufficient to achieve treatment, as defined herein, of Parkinson's Disease, as compared to the response obtained without administration of the agent.

Encapsulate: As used herein, the term “encapsulate” means to enclose, surround, or encase.

Engineered: As used herein, embodiments are “engineered” when they are designed to have a feature or property, whether structural or chemical, that varies from a starting point, wild-type or native molecule. Thus, engineered agents or entities are those whose design and/or production include an act of the hand of man.

Epitope: As used herein, an “epitope” refers to a surface or region on a molecule that is capable of interacting with a biomolecule. For example, a protein may contain one or more amino acids, e.g., an epitope, which interacts with an antibody, e.g., a biomolecule. In some embodiments, when referring to a protein or protein module, an epitope may comprise a linear stretch of amino acids or a three-dimensional structure formed by folded amino acid chains.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

In some embodiments, antigenic material may be used or produced for inclusion in the compositions, and the antigen can be in the form of a protein, glycoprotein, or antigenic oligosaccharide, which may, in some embodiments represent an antigen from a virus, bacterium, fungus, or protozoan. In some embodiments, the antigenic material is a synthetically produced antigenic material. For example, the pan-fungal biomarker 1-3 beta-d-glucan (also known as (1->3)-β-D-Glucan, or “BDG”) is a polysaccharide composed of glucose monomers linked by beta 1-3 glucosidic bonds and is found in high abundance in cellulose-containing plants, and in the cell wall of many fungi. Because yeast-derived β-glucans (such as β-1,3/1,6-glucan derived from baker's yeast) have been reported to stimulate the immune system, they are commercially available and marketed as a dietary supplement. In some embodiments, the synthetically produced antigenic material is made by recombinant DNA technology. In some embodiments, the antigenic material is an mRNA vaccine, which is administered to a subject to express the encoded protein or antigenic material in the subject.

In some embodiments, the antigenic material is, or is from, a virus. In some embodiments, the antigen material is an attenuated virus, an inactivated virus, a viral protein, recombinant viral protein, or mRNA vaccine of a viral protein. In some embodiments, the antigenic material from a virus is a viral toxin. In some embodiments, the viral toxin is rotavirus NSP4.

In some embodiments, the antigenic material is, or is from, a bacterium. In some embodiments, the antigenic material is attenuated or killed bacteria. In some embodiments, the antigenic material comprises two or more bacterial strains or species. In some embodiments, the antigenic material is a conjugate of two or more bacterial proteins and/or bacterial polysaccharides. In some embodiments, the antigenic material from bacteria is a bacterial toxin or subunit vaccine. In some embodiments, the antigenic material is an mRNA vaccine of a bacterial protein.

In some embodiments, the antigenic material is, or is from, a fungal pathogen. In some embodiments, the antigenic material is, or is from, pathogenic Coccidioides, Aspergillus, Histoplasma, Blastomyces, Paracoccidioides, Pneumocystis, or Cryptococcus. In some embodiments, the antigenic material is an mRNA vaccine of a fungal protein.

In some embodiments, the antigenic material is, or is from, a protozoal pathogen. In some embodiments, the antigenic material is, or is from, Eimeria, Theileria, Toxoplasma, Tritrichomonas, Giardia, Sarcocystis, Neospora, Leishmania, or Babesia. In some embodiments, the antigenic material comprises a carbohydrate/polysaccharide antigen of a protozoal pathogen. In some embodiments, the antigenic material is an mRNA vaccine of a protozoal protein.

In some embodiments, the therapeutic SynC composition is administered prior to, concurrently with, or subsequent to administration of an antigenic material or vaccine. In some embodiments, the therapeutic SynC composition is administered concurrently with the antigenic material or vaccine, either separately by the same or different administration route, or together as a single composition. In some embodiments, the therapeutic SynC composition is subsequently administered to the subject one or more times following an initial vaccination. In some embodiments, the administration of the therapeutic SynC composition is performed without a subsequent dose of the antigenic material or vaccine. In some embodiments, following initial vaccination with antigenic material or vaccine with or without the presently disclosed therapeutic SynC composition, one or more doses of the therapeutic SynC compositions are administered and followed by a second vaccination with antigenic material or vaccine, with or without the therapeutic SynC composition.

Feature: As used herein, a “feature” refers to a characteristic, a property, or a distinctive element.

Formulation: As used herein, a “formulation” includes at least one polynucleotide and/or compound and/or composition of the present disclosure (e.g., a platform SynC, cells containing a bioengineered mSynC with integrated gene(s), etc.) and a delivery agent.

Fragment: A “fragment,” as used herein, refers to a contiguous portion of a whole. For example, fragments of proteins may comprise polypeptides obtained by digesting full-length protein isolated from cultured cells. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. In some embodiments, fragments of an antibody include portions of an antibody subjected to enzymatic digestion or synthesized as such.

Functional: As used herein, a “functional” biological molecule is a biological molecule and/or entity with a structure and in a form in which it exhibits a property and/or activity by which it is characterized.

As used herein, the word “gene” includes native genomic nucleic acid sequences as they are found at a chromosomal locus, or “gene” can be used to refer to a nucleic acid sequence that includes both introns and exons. or can be used to refer to the nucleic acid sequence that encodes a messenger RNA (mRNA), or can refer to a recombinant RNA that includes only exons, such as a cDNA, Thus, “gene(s)” can also refer to those nucleic acid sequences that are “expressed” by getting transcribed into RNA molecules such as mRNA, a tRNA, an rRNA, or other functional RNAs, such as small nucleolar RNAs (snoRNAs), and the like. Further, a gene may be transcribed and translated into a peptide, polypeptide or protein. A “gene product” can refer to a protein, a polypeptide or a small peptide, or to RNAs which may be transcribed, and may or may not be translated; thus, gene products include functional RNAs, small nuclear RNAs (snRNAs), small nucleolar RNAs (snoRNAs), for example. In some embodiments of the present compositions and methods, a gene is found within a “cassette” that has been engineered to include a length of nucleic acid sequence designed to comprise one or more functional RNAs and/or expressible sequences that in some cases are transcribed into functional gene products such as RNAs and may be translated into gene products that are proteins. Thus, “gene(s)” includes genomic DNAs, mRNAs, tRNAs and rRNA, cDNAs, coding sequences with or without introns, may encode single or multiple, exons, as well as only a partial sequence translated into a partial protein, or any combination of these, (e.g., multiple linked genes comprising genomic sequences linked to a cDNA sequence) such as in a cassette.

A “large gene” is meant to refer to a nucleic acid sequence greater than average in sequence length. Thus, when referring to a genomic sequence comprising a gene, a large genomic sequence means a genomic sequence larger than average/ordinary. Similarly, when referring to a large coding sequence in a cDNA, a large coding sequence means larger than most coding sequences. An example of an extraordinarily large gene in mammals is the dystrophin gene. In some embodiments, a large genomic fragment is contained in a bacterial artificial chromosome, and cannot necessarily be contained by an alternate vector type, such as a plasmid or viral vector. In some embodiments, a large genomic region can span chromosome specific banding regions.

As used herein, especially when referring to a nucleic acid sequence to be inserted into a synthetic chromosome, the phrases “large genetic payloads,” “a large nucleic acid sequence,” “a large genomic DNA,” “a large cDNA” and “a large gene,” refer to sequences greater than the carrying capacity of a viral-based vector, such as the AAV vectors and usually employed in presently available gene therapies (which AAV vectors have a carrying capacity of approximately 5 kbps in length as the upper limit). In the present compositions and methods, “large” sequences can be many megabasepairs (Mbps) in length.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, 96%, 97%, 98%, 99% or 100% identical or similar. The term “homologous” necessarily refers to a comparison of and an amount of similarity between at least two sequences (polynucleotide or polypeptide sequences). In accordance with the invention, two polynucleotide sequences are considered to be homologous if the polypeptides they encode are at least about 50%, 60%, 70%, 80%, 90%, 92%, 93%, 94% 95%, 96%, 97%, 98%, 99%, 99.5%, 99.6%, 99.7%, 99.8% or even 99.9% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is typically determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids. In many embodiments, homologous protein may show a large overall degree of homology and a high degree of homology over at least one short stretch of at least 3, 4, 5, 6, 7, 8, 9, 10, 11, 12, 13, 14, 15, 16, 17, 18, 19, 20, 25, 30, 35, 40, 45, 50 or more amino acids. In many embodiments, homologous proteins share one or more characteristic sequence elements. As used herein, the term “characteristic sequence element” refers to a motif present in related proteins. In some embodiments, the presence of such motifs correlates with a particular activity (such as biological activity).

With reference to comparison of sequences (nucleic acid or polypeptide) for evolutionary relatedness and conservation of activity/function, the phrase “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). While all of the above-mentioned algorithms and programs are suitable for a determination of sequence alignment and % sequence identity, for purposes of the disclosure herein, determination of % sequence identity will typically be performed using the BESTFIT or GAP programs in the GCG Wisconsin Software package (Accelrys, Madison WI), using default parameters provided.

Identity: As used herein, the term “identity” refers to the overall relatedness between polymeric molecules, e.g., between oligonucleotide and/or polynucleotide molecules (e.g. DNA molecules and/or RNA molecules) and/or between polypeptide molecules. Calculation of the percent identity of two polynucleotide sequences, for example, may be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference in its entirety. For example, the percent identity between two nucleotide sequences can be determined, for example using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference in its entirety. Techniques for determining identity are codified in publicly available computer programs. Exemplary computer software to determine homology between two sequences include, but are not limited to, GCG program package, Devereux, J., et al., Nucleic Acids Research, 12 (1), 387 (1984)), BLASTP, BLASTN, and FASTA Altschul, S. F. et al. J. Molec. Biol., 215, 403 (1990)).

Inhibit expression of a gene: As used herein, the phrase “inhibit expression of a gene” means to cause a reduction in the amount of an expression product of the gene. The expression product may be RNA transcribed from the gene (e.g. mRNA) or a polypeptide translated from mRNA transcribed from the gene. Typically, a reduction in the level of mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” is synonymous with “separated”, but carries with it the inference separation was carried out by the hand of man. In one embodiment, an isolated substance or entity is one that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: As used herein, “substantially isolated” means that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art. In some embodiments, isolation of a substance or entity includes disruption of chemical associations and/or bonds. In some embodiments, isolation includes only the separation from components with which the isolated substance or entity was previously combined and does not include such disruption.

Modified: As used herein, the term “modified” refers to a changed state or structure of a molecule or entity of the invention as compared with a parent or reference molecule or entity. Molecules may be modified in many ways including chemically, structurally, and functionally. In some embodiments, compounds and/or compositions of the present disclosure are modified by the introduction of non-natural amino acids, or non-natural nucleotides.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids). In embodiments wherein mutations comprise the addition and/or substitution of amino acids and/or nucleotides, such additions and/or substitutions may comprise 1 or more amino acid and/or nucleotide residues and may include modified amino acids and/or nucleotides.

Naturally occurring: As used herein, “naturally occurring” means existing in nature without artificial aid or involvement of the hand of man

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic acid: As used herein, the term “nucleic acid”, “polynucleotide” and ‘oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA.

Off-target: As used herein, “off target” refers to any unintended effect on any one or more target, gene and/or cellular transcript.

Open reading frame: As used herein, “open reading frame” or “ORF” refers to a sequence which does not contain a stop codon in a given reading frame.

Operably linked: As used herein, the phrase “operably linked” refers to a functional connection between two or more molecules, constructs, transcripts, entities, moieties or the like.

Particle: As used herein, a “particle” is a virus having at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained professional for a particular disease or condition, such as for example Parkinson's Disease.

Payload: As used herein, “payload” refers to one or more polynucleotides or polynucleotide regions encoded by or within an mSynC or in an expression cassette, and may also refer to a product of such polynucleotide or polynucleotide region, e.g., a transgene or transgene mRNA encoded by a transgene. In some instances, a payload could be an encoded peptide, polypeptide or multi-polypeptide, or a modulatory nucleic acid or regulatory nucleic acid being delivered to a target cell, tissue, organism, animal, mammal, or human subject. In some instances, a synthetic chromosome can be engineered to carry a large genetic payload encoding phenotypic traits that are normally unlinked in the native genomic environment of a host animal; the use of a synthetic chromosome allows the conveyance of a new repertoire of multigenic traits to be brought together in a stable, non-integrating, self-replicating, and portable bioengineering system.

As used herein, a “delivery vector,” (which may also be referred to as a “genetic payload construct”) can be a plasmid vector or bacterial artificial chromosome (BAC) or a yeast artificial chromosome (YAC) bearing one or more polynucleotide regions encoding or comprising a genetic payload, with or without regulatory regions, promoters, genetic enhancer sequences and the like (which can be referred to as a “cassette”) for delivery to the mSynC, for example.

Peptide: As used herein, “peptide” is less than or equal to approximately 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier” or “pharmaceutically acceptable excipient” refers to an excipient, carrier or adjuvant that can be administered to a subject, together with at least one therapeutic agent, and which does not destroy the pharmacological activity thereof and is generally safe, nontoxic and neither biologically nor otherwise undesirable when administered in doses sufficient to deliver a therapeutic amount of the agent.

“Adjuvant” refers to a substance or combination of substances that enhances a subject's response to a therapeutic agent (e.g., by stimulating a stronger immune response in people receiving a vaccine). A vaccine adjuvant assists the vaccine, allowing it to achieve the same efficacy even when the dosage amount and/or frequency is reduced, thereby helping to minimize any potential side effects. Adjuvanted vaccines can, in some cases, cause more local reactions (such as redness, swelling, and pain at the injection site) and more systemic reactions (such as fever, chills and body aches) than non-adjuvanted vaccines. Some vaccines made from weakened or killed pathogens contain naturally occurring adjuvants and help the body produce a strong protective immune response. Adjuvants that stimulate immune activation of several innate pathways and adaptive immune systems simultaneously can induce more robust immune responses (both humoral and cellular adaptive immune responses), thus having a great potential to be successful in the clinic, particularly in patients with weaker immune systems.

Pharmaceutically acceptable excipients: The phrase “pharmaceutically acceptable excipient,” as used herein, refers to any ingredient other than the compounds and/or active agents (e.g. as described herein) present in pharmaceutical compositions and having the properties of being substantially nontoxic and non-inflammatory in a subject such as a patient. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspension or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystal line cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The phrase “pharmaceutically acceptable salts,” as used in reference to the compounds described herein, refers to derivatives or forms of the disclosed compounds, wherein the parent compound is modified by converting an existing acid or base moiety to its salt form (e.g., as generated by reacting the free base group with a suitable organic acid). The phrase is meant to include salts of the active compounds which are prepared with relatively nontoxic acids or bases, depending on the particular substituents found on the compounds described herein. Examples of pharmaceutically acceptable salts include, but are not limited to, mineral or organic acid salts of basic residues such as amines; alkali or organic salts of acidic residues such as carboxylic acids; and the like. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, phosphoric, partially neutralized phosphoric acids, sulfuric, partially neutralized sulfuric, hydroiodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure may contain both basic and acidic functionalities that allow the compounds to be converted into either base or acid addition salts. Lists of suitable salts are found in Remington's Pharmaceutical Sciences, 17th Ed., Mack Publishing Company, Easton, Pa., (1985) and Journal of Pharmaceutical Science, 66:2 (1977), each of which is incorporated herein by reference in its entirety.

Representative acid addition salts include acetate, acetic acid, adipate, alginate, ascorbate, aspartate, benzenesulfonate, benzene sulfonic acid, benzoate, bisulfate, borate, butyrate, camphorate, canwhorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, laurel sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. In some embodiments a pharmaceutically acceptable salt of the present disclosure can be synthesized salt prepared from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

“Solvate” refers to a complex of variable stoichiometry formed by a solute, such as a drug, compound or multi-component composition described herein, and a solvent. Such solvents are selected to minimally interfere with the biological activity of the solute. Solvents may be, by way of example and not limitation, water, ethanol, or acetic acid.

Pharmaceutically acceptable solvate: The term “pharmaceutically acceptable solvate,” as used herein, refers to a crystalline form of a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (MIR), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition, such as for example Parkinson's Disease.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may by covalently bonded or sequestered in some way and which release or are converted into the active drug moiety prior to, upon or after administered to a mammalian subject. Preparation and use of prodrugs is discussed in Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety. “Prodrug” refers to a derivative of an active compound (e.g., drug) that requires a transformation under the conditions of use, such as within the body or appropriate in vitro conditions, to release the active drug. Prodrugs are frequently, but not necessarily, pharmacologically inactive until converted into the active drug. In some embodiments of the present disclosure, an initial transcript or gene product might be considered a “prodrug” in that it might undergo a transformation in part required for activity with a progroup to form a promoiety, (e.g., acetylation, methylation, histone modification, glycosylation, cleavage of a nucleic acid or protein gene product, or other modification) to make the gene product functional in a certain environment, such as cells or tissue in vitro, ex vivo, or in vivo, such as in a mammal. The cleavage of the promoiety may proceed spontaneously, such as by way of a hydrolysis reaction, or it may be catalyzed or induced by another agent, such as by an enzyme, by light, by acid, or by a change of or exposure to a physical or environmental parameter, such as a change of temperature. The agent may be endogenous to the conditions of use, such as an enzyme present in the cells to which the prodrug is administered or the acidic conditions of the stomach, or it may be supplied exogenously.

Proliferate: As used herein, the term “proliferate” means to grow, expand, replicate or increase or cause to grow, expand, replicate or increase. “Proliferative” means having the ability to proliferate. “Anti-proliferative” means having properties counter to or in opposition to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” include those provided herein and fragments, mutants, variants, and alterations thereof.

For example, a variant of a nucleic acid or a protein would be expected to share less than 100% sequence identity but would have enough homology to indicate significant conservation of function/activity.

Purified: As used herein, “purify,” “purified,” “purification” means to make substantially pure or clear from unwanted components, material defilement, admixture or imperfection. “Purified” refers to the state of being pure. “Purification” refers to the process of making pure.

A protein “domain” as used herein refers to an amino acid sequence of a chimeric polypeptide comprising one or more defined functions or properties.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may therefore comprise the N- and/or C-termini as well as surrounding amino acids. In some embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may therefore comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus but does not include the 3′ terminus. In some embodiments, 3′ regions may comprise any length of nucleic acids, which include the 3′ terminus, but does not comprise the 5′ terminus.

RNA interference: As used herein, the term “RNA interference” or “RNAi” refers to a sequence specific regulatory mechanism mediated by RNA molecules which results in the inhibition or interference or “silencing” of the expression of a corresponding protein-coding gene.

Sample: As used herein, the term “sample” refers to an aliquot, subset or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, or organs. In some embodiments, a sample is or comprises a medium, such as a nutrient broth or gel, which may contain cellular components, such as proteins or nucleic acid molecules. In some embodiments, a “primary” sample is an aliquot of the source. In some embodiments, a primary sample is subjected to one or more processing (e.g., separation, purification, etc.) steps to prepare a sample for analysis or other use.

Sense strand: As used herein, the term “the sense strand” or “the second strand” or “the passenger strand” of a siRNA molecule refers to a strand that is complementary to the anti-sense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the siRNA strand.

Signal Sequences: As used herein, the phrase “signal sequences” refers to a sequence which can direct the transport or localization.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Small/short interfering RNA: As used herein, the term “small/short interfering RNA” or “siRNA” refers to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Split dose: As used herein, a “split dose” is the division of single unit dose or total daily dose into two or more doses.

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize,” “stabilized,” “stabilized region” means to make or become stable. In some embodiments, stability is measured relative to an absolute value. In some embodiments, stability is measured relative to a reference compound or entity.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, the subject may be an infant, neonate, or a child under the age of 12 years old. In some embodiments, the subject may be in utero.

The terms “subject,” “individual,” “host” or “patient” may be used interchangeably herein and typically refer to a vertebrate, most typically a mammal. In some embodiments, the subject is murine, rodent, lagomorph, feline, canine, porcine, ovine, bovine, equine, or primate. In some embodiments, the subject is a mammal. Appropriate subjects may also include, but are not limited to, rodents (mice, rats, etc.), simians, humans, mammalian farm animals, mammalian sport animals, and mammalian pets, but can also include commercially relevant birds such as chickens, ducks, geese, quail, and/or turkeys. A mammalian subject may be human or other primate (e.g., cynomolgus monkey, rhesus monkey), or commercially relevant mammals, farm animals, sport animals, and pets such as cattle, pigs, horses, sheep, goats, rabbits and hares, cats, and/or dogs. In some embodiments, the subject is a non-human primate, for example a monkey, chimpanzee, or gorilla. In some embodiments, the subject is a human, sometimes referred to herein as a patient. In some embodiments, the subject is female. In some embodiments, the subject is male. In some embodiments, the subject is intersex. The subject can be a male or female of any age group, e.g., a pediatric subject (e.g., infant, child, adolescent) or adult subject (e.g., young adult, middle-aged adult or senior adult). In some embodiments, the subject may be an infant, child, adolescent or adult.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term typically means within about 2 seconds.

Suffering from: An individual who is “suffering from” a disease, disorder, and/or condition has been diagnosed with or displays one or more symptoms of a disease, disorder, and/or condition such as for example Parkinson's Disease.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

As used herein, a “switch” refers to a regulatory mechanism for turning on and/or off the synthetic chromosome (all or part of the synthetic chromosome itself), and/or a mechanism for specifically eliminating host cells carrying the synthetic chromosome by shunting the host cells down an apoptotic pathway. As used herein, a switch can be a genetic sequence itself under tightly regulatable control (e.g., by a very efficient, strictly controllable promoter) wherein the switch encodes one or more gene product(s) that can directly or indirectly regulate the presence and/or expression of one or more target genes, or the switch may regulate survival of a host cell carrying the synthetic chromosome. The target genes being regulated by the switch gene products may be themselves carried on the synthetic chromosome, or the target genes may be encoded in a host cell genome. A switch may be designed to turn on and/or off expression of target genes on the synthetic chromosome or in host cells or may be designed to eliminate the synthetic chromosome-carrying host cells themselves. One example of a switch can be a silencing switch that can regulate heterochromatinization of the synthetic chromosome, specifically, allowing regulation of expression of genes encoded on the synthetic chromosome or directly or indirectly regulating genes in the host cell genome. Another example of a switch can make use of apoptotic pathways, and may employ a regulatable and tunable balance of pro- and anti-apoptotic factors.

SynC broadly means Synthetic Chromosome engineered by a method based on the SATAC method and engineered to contain multiple, site-specific recombination sites into which exogenous DNA elements may be integrated. An mSynC means a mammalian SynC.

Synthetic: The term “synthetic” means produced, prepared, and/or manufactured by the hand of man. Synthesis of polynucleotides or polypeptides or other molecules of the present invention may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition such as for example Parkinson's Disease. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in a 24-hour period. It may be administered as a single unit dose.

A “vector” is a replicon, such as plasmid, phage, viral construct, cosmid, bacterial artificial chromosome, P-1 derived artificial chromosome or yeast artificial chromosome to which another DNA segment may be attached. In some instances, a vector may be a chromosome such as in the case of an arm exchange from one endogenous chromosome engineered to comprise a recombination site to a synthetic chromosome. Vectors are used to transduce and express a DNA segment in a cell. In some embodiments, a delivery vector is used to introduce an expression cassette onto the synthetic platform chromosome. The delivery vector may include additional elements; for example, the delivery vector may have one or two replication systems, thus allowing it to be maintained in organisms, for example in mammalian cells for expression and in a prokaryotic host for cloning and amplification.

“Site-specific recombination” refers to site-specific recombination that is effected between two specific sites on a single nucleic acid molecule or between two different molecules that requires the presence of an exogenous protein, such as an integrase or recombinase. Certain site-specific recombination systems can be used to specifically delete, invert, or insert DNA, with the precise event controlled by the orientation of the specific sites, the specific system and the presence of accessory proteins or factors. In addition, segments of DNA can be exchanged between chromosomes, such as in chromosome arm exchange.

The choice of delivery vector to be used to deliver or “load” the multiple regulatory control systems and multiple genes onto the synthetic platform chromosome will depend upon a variety of factors such as the type of cell in which propagation is desired. The choice of appropriate delivery vector is well within the skill of those in the art, and many vectors are available commercially. To prepare the delivery vector, one or more genes under the control of one or more regulatory control systems are inserted into a vector, typically by means of ligation of the gene sequences into a cleaved restriction enzyme site in the vector. The delivery vector and the desired multiple regulatory control systems may also be synthesized in whole or in fractions that are subsequently connected by in vitro methods known to those skilled in the art. Alternatively, the desired nucleotide sequences can be inserted by homologous recombination or site-specific recombination. Typically, homologous recombination is accomplished by attaching regions of homology to the vector on the flanks of the desired nucleotide sequence (e.g., cre-lox, att sites, etc.). Nucleic acids containing such sequences can be added by, for example, ligation of oligonucleotides, or by polymerase chain reaction using primers comprising both the region of homology and a portion of the desired nucleotide sequence. Exemplary delivery vectors that may be used include but are not limited to those derived from recombinant bacteriophage DNA, plasmid DNA or cosmid DNA. For example, plasmid vectors such as pBR322, pUC 19/18, pUC 118, 119, and the M13 mp series of vectors may be used. Bacteriophage vectors may include Agt10, 2gt11, 2gt18-23, AZAP/R and the EMBL series of bacteriophage vectors. Cosmid vectors that may be utilized include, but are not limited to, pJB8, pCV 103, pCV 107, pCV 108, pTM, pMCS, pNNL, pHSG274, COS202, COS203, pWE15, pWE16 and the charomid 9 series of vectors. Additional vectors include bacterial artificial chromosomes (BACs) based on a functional fertility plasmid (F-plasmid), yeast artificial chromosomes (YACs), and P1-derived artificial chromosomes, DNA constructs derived from the DNA of P1 bacteriophage (PACS). Alternatively, recombinant virus vectors may be engineered, including but not limited to those derived from viruses such as herpes virus, retroviruses, vaccinia virus, poxviruses, adenoviruses, lentiviruses, adeno-associated viruses or bovine papilloma virus. Alternatively, the genes under control of the regulatory control systems may be loaded onto the synthetic platform chromosome via sequential loading using multiple delivery vectors; that is, a first gene under control of a first regulatory control system may be loaded onto the synthetic platform chromosome via a first delivery vector, a second gene under control of a second regulatory control system may be loaded onto the synthetic platform chromosome via a second delivery vector, and so on. Perkins and Greene, U.S. Ser. No. 62/321,711 filed 12 Apr. 2016, describe sequential loading of genes onto a synthetic platform chromosome using multiple delivery vectors while recycling a single selectable marker.

Using lambda integrase mediated site-specific recombination—or any other recombinase-mediated site-specific recombination—the genes under regulatory control are introduced or “loaded” from the delivery vector onto the synthetic platform chromosome. Because the synthetic platform chromosome contains multiple site-specific recombination sites, the multiple genes may be loaded onto a single synthetic platform chromosome. The recombinase that mediates the site-specific recombination may be delivered to the cell by encoding the gene for the recombinase on the delivery vector, or purified protein or encapsulated recombinase protein delivered to a recipient cell using standard technologies. Each of the multiple genes may be under the control of its own regulatory control system; alternatively, the expression of the multiple genes may be coordinately regulated via viral-based or human internal ribosome entry site (IRES) elements (see, e.g., Jackson et al., Trends Biochem Sci. 15:477-83 (1990); and Oumard et al., Mol. Cell. Biol. 20:2755-2759 (2000)) or 2A self-cleaving peptides (See, Kim, et al., PLOS ONE, 6 (4), e18556. Online at //doi.org/10.1371/journal.pone.0018556) or as pro-peptides responsive to the host cells endogenous processing system (e.g., preproinsulin, Liu, et al., Diabetes Obes. Metabol. Suppl. 2:28-50). Additionally, using IRES type elements or 2A peptides linked to a fluorescent marker downstream from the target genes—e.g., green, red or blue fluorescent proteins (GFP, RFP, BFP)—allows for the identification of synthetic platform chromosomes expressing the integrated target genes. Alternatively, or in addition, site-specific recombination events on the synthetic chromosome can be quickly screened by designing primers to detect integration by PCR.

Transfection: As used herein, the term “transfection” refers to methods to introduce exogenous nucleic acids into a cell. Methods of transfection include, but are not limited to, chemical methods, physical treatments and cationic lipids or mixtures. The vectors carrying the components appropriate for synthetic chromosome production can be delivered to the cells to produce the synthetic chromosome by any method known in the art. The terms transfection and transformation refer to the taking up of exogenous nucleic acid, e.g., an expression vector, by a host cell whether or not any coding sequences are, in fact, expressed. Numerous methods of transfection are known to the ordinarily skilled artisan, for example, by Agrobacterium-mediated transformation, protoplast transformation (including polyethylene glycol (PEG)-mediated transformation, electroporation, protoplast fusion, and microcell fusion), lipid-mediated delivery, liposomes, electroporation, sonoporation, microinjection, particle bombardment and silicon carbide whisker-mediated transformation and combinations thereof (see, e.g., Paszkowski, et al., EMBO J., 3:2717-2722 (1984); Potrykus, et al., Mol. Gen. Genet., 199:169-177 (1985); Reich, et al., Biotechnology, 4:1001-1004 (1986); Klein, et al., Nature, 327:70-73 (1987); U.S. Pat. No. 6,143,949; Paszkowski, et al., in Cell Culture and Somatic Cell Genetics of Plants, Vol. 6, Molecular Biology of Plant Nuclear Genes, (Schell and Vasil, eds., Academic Publishers 1989); and Frame, et al., Plant J., 6:941-948 (1994)); direct uptake using calcium phosphate (Wigler, et al., Proc. Natl. Acad. Sci. U.S.A., 76:1373-1376 (1979)); polyethylene glycol (PEG)-mediated DNA uptake; lipofection (see, e.g., Strauss, Meth. Mol. Biol., 54:307-327 (1996)); microcell fusion (Lambert, Proc. Natl. Acad. Sci. U.S.A., 88:5907-5911 (1991); U.S. Pat. No. 5,396,767; Sawford, et al., Somatic Cell Mol. Genet., 13:279-284 (1987); Dhar, et al., Somatic Cell Mol. Genet., 10:547-559 (1984); and McNeill-Killary, et al., Meth. Enzymol., 254:133-152 (1995)); lipid-mediated carrier systems (see, e.g., Teifel, et al., Biotechniques, 19:79-80 (1995); Albrecht, et al., Ann. Hematol., 72:73-79 (1996); Holmen, et al., In Vitro Cell Dev. Biol. Anim., 31:347-351 (1995); Remy, et al., Bioconjug. Chem., 5:647-654 (1994); Le Bolch, et al., Tetrahedron Lett., 36:6681-6684 (1995); and Loeffler, et al., Meth. Enzymol., 217:599-618 (1993)); or other suitable methods. Methods for delivery of synthetic chromosomes also are described in U.S. application Ser. No. 09/815,979. Successful transfection is generally recognized by detection of the presence of the heterologous nucleic acid within the transfected cell, such as, for example, any visualization of the heterologous nucleic acid, expression of a selectable marker or any indication of the operation of a vector within the host cell. For a description of delivery methods useful in practicing the present invention, see U.S. Pat. Nos. 5,011,776; 5,747,308; 4,966,843; 5,627,059; 5,681,713; Kim and Eberwine, Anal. Bioanal. Chem. 397 (8): 3173-3178 (2010).

Treating: As used herein, the term “treating” refers to partially or completely alleviating, ameliorating, improving, relieving, delaying onset of, inhibiting and/or slowing the progression of, reducing severity of, and/or reducing incidence of one or more symptoms or features of a particular infection, disease, disorder, and/or condition. Treating can mean modifying the disease process, such as in “treating” cancer by inhibiting survival, growth, and/or spread of a tumor, or by relieving current symptoms, slowing or inhibiting the development of new symptoms. Treating may also mean prolonging the life of a diseased patient. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition such as, for example, Parkinson's Disease or muscular dystrophy.

Unmodified: As used herein, “unmodified” refers to any substance, compound or molecule prior to being changed in any way. Unmodified may, but does not always, refer to the wild type or native form of a biomolecule or entity. Molecules or entities may undergo a series of modifications whereby each modified substance, compound, molecule or entity may serve as the “unmodified” starting molecule for a subsequent modification.

Vector: As used herein, a “vector” is any molecule or moiety which transports, transduces or otherwise acts as a carrier of a heterologous molecule. Vectors of the present disclosure may be produced recombinantly.

### Visualization, Isolation, and Transfer to Recipient Immune Cells

The production and loading of the synthetic platform chromosomes of the present invention can be monitored by various methods. Lindenbaum, M., Perkins, E., et al., Nucleic Acid Research, 32 (21): e172 (2004) describe the production of a mammalian satellite DNA-based Artificial Chromosome Expression (ACE) System. In this system, conventional single-color and two-color FISH analysis and high-resolution FISH were carried out using PCR-generated probes or nick-translated probes. For detection of telomere sequences, mitotic spreads were hybridized with a commercially obtained peptide nucleic acid probe. Microscopy was performed using fluorescent microscopy. Alternatively, Perkins and Greene, PCT/US16/17179 filed 9 Feb. 2016, describes compositions and methods to allow one to monitor formation of synthetic chromosomes in real-time via standardized fluorescent technology using two labeled tags: one labeled tag specific to endogenous chromosomes in the cell line used to produce the synthetic platform chromosomes, and one differently-labeled tag specific to a sequence on the synthetic chromosome that is to be produced.

Isolation and transfer of synthetic chromosomes typically involves utilizing microcell mediated cell transfer (MMCT) technology or dye-dependent, chromosome staining with subsequent flow cytometric-based sorting. In the MMCT technique, donor cells are chemically induced to multinucleate their chromosomes with subsequent packaging into microcells and eventual fusion into recipient cells. Establishing that the synthetic chromosomes have been transferred to recipient cells is carried out with drug selection and intact delivery of the transferred chromosome confirmed by FISH. Alternatively, flow cytometric-based transfer can be used. For flow cytometric-based transfer, mitotically arrested chromosomes are isolated and stained with DNA specific dyes and flow sorted based on size and differential dye staining. The flow-sorted chromosomes are then delivered into recipient cells via standard DNA transfection technology, and delivery of intact chromosomes is determined by FISH or Flow-FISH (Rigolin, et al., 2004, Eur. J. Haemotol., 73 (5): 351-8). In yet another alternative, in addition to the visualization and monitoring of synthetic chromosome production described in Perkins and Greene, PCT/US16/17179 filed 9 Feb. 2016, the synthetic chromosome tags can be used to isolate the synthetic chromosomes from the synthetic chromosome production cells via flow cytometry, as well as to monitor the transfer of the synthetic chromosomes into recipient cells.

### Transforming Mammalian Target Cells

To date, isolation and transfer of artificial chromosomes has involved utilizing microcell mediated cell transfer (MMCT) technology or dye-dependent chromosome staining with subsequent flow cytometric-based sorting. In the MMCT technique, donor cells are chemically induced to multinucleate their chromosomes with subsequent packaging into microcells and eventual fusion into recipient cells. The establishment of transferred chromosomes in the recipient cells is carried out with drug selection and intact delivery of the transferred chromosome confirmed by FISH. For flow cytometric-based transfer, mitotically arrested chromosomes are isolated and stained with DNA specific dyes or DNA sequence-specific probes or DNA sequence-specific engineered proteins such as native repressors (e.g. lac repressor), TALON engineered proteins, CRISPR-Cas9 derivatives, and engineered Zn-finger nucleases. Using these methods, the synthetic chromosomes can be simply flow-sorted based on size and differential dye staining, and the flow-sorted chromosomes are then delivered into recipient cells via standard DNA transfection technology, and delivery of intact chromosomes is determined by FISH or Flow-FISH.

Peptide nucleic acids (PNAs) are an artificially synthesized polymer similar to DNA or RNA. Commercially available fluorescently labeled PNAs can be used to visualize the hSynCs of the present disclosure. For example, New England Biolabs (NEB®) offers a selection of fluorescent labels (substrates) for SNAP- and CLIP-tag fusion proteins. SNAP-tag® substrates consist of a fluorophore conjugated to guanine or chloropyrimidine leaving groups via a benzyl linker, while CLIP-tag™ substrates consist of a fluorophore conjugated to a cytosine leaving group via a benzyl linker. These substrates will label their respective tags without the need for additional enzymes. Cell-permeable substrates (SNAP-Cell® and CLIP-Cell™) are suitable for both intracellular and cell-surface labeling, whereas non-cell-permeable substrates (SNAP-Surface® and CLIP-Surface™) are specific for fusion proteins expressed on the cell surface only.

As an alternative, CRISPR editing technologies can be adapted to visualize the synthetic chromosomes and to isolate and purify the synthetic chromosomes prior to delivery to target cells. (See co-pending patent application PCT/US16/17179.) In this process, unique DNA elements/sequences are incorporated into the synthetic chromosomes during production in the synthetic chromosome production cells. The presence of these unique DNA elements/sequences on the synthetic chromosome permits specific targeting of an engineered, nuclease deficient CRISPR/Cas-fluorescent protein visualization complex (CRISPR/CAS-FP) directly to the synthetic chromosome without binding to native, endogenous chromosomes. Subsequently, the binding of the CRISPR/CAS-FP to the synthetic chromosome provides a means to purify the synthetic chromosome by flow cytometry/flow sorting for eventual delivery into recipient cells. The synthetic chromosome production cells are subjected to mitotic arrest followed by purification of the synthetic chromosome by flow cytometry/flow sorting based on the unique CRISPR-fluorescent tag binding to the synthetic chromosome.

The use of CRISPR/CAS-FP bypasses the need for using potentially mutagenic chromosome dyes and alleviates the potential contamination of dye-stained endogenous chromosomes contaminating preparations of flow-sorted synthetic chromosomes. In addition, purified synthetic chromosomes bound with CRISPR/Cas-FP can be utilized for assessing the efficiency of delivery of flow-sorted synthetic chromosomes into recipient target cells by simple measurement of fluorescent signal quantity in a transfected recipient cell population. The CRISPR/Cas-FP bound synthetic chromosomes also can be utilized to flow sort purify or enrich for synthetic chromosome transfected cells. Fluorescent proteins of particular use include but are not limited to TagBFP, TagCFP, TagGFP2, TagYFP, TagRFP, FusionRed, mKate2, TurboGFP, Turbo YFP, TurboRFP, TurboFP602, TurboFP635, or TurboFP650 (all available from Evrogen, Moscow); AmCyan1, AcvGFP1, ZsGreen1, ZsYellow1, mBanana, mOrange, mOrange2, DsRed-Express2, DsRed-Express, tdTomato, DsRed-Monomer, DsRed2, AsRed2, mStrawberry, mCherry, HcRed1, mRaspberry, E2-Crimson, mPlum, Dendra 2, Timer, and PAmCherry (all available from Clontech, Palo Alto, CA); HALO-tags; infrared (far red shifted) tags (available from Promega, Madison, WI); and other fluorescent tags known in the art, as well as fluorescent tags subsequently discovered. For example, in some embodiments, SNAP-tags may be used to identify transfected cells following transfection.

In some embodiments, to address safety of the system in clinical applications and to be able to shut off the system once a therapeutic endpoint was reached, a safety switch was designed and installed on the synthetic chromosome to regulate the activity of one or more genes encoded upon and/or expressed from the synthetic chromosome.

A safety switch could comprise nucleic acid sequences encoding or regulating expression of one or more pro-apoptotic proteins or regulatory nucleic acids. In some embodiments, one or more genes may be present on the synthetic chromosome, or may be engineered into the target cell intended to carry the synthetic chromosome, to encode counterbalancing anti-apoptotic proteins or regulatory nucleic acids.

Progress in bioengineering of cells for gene-based therapies has been held back by the absence of an indispensable tool required to address complex polygenicity and/or delivery of large genetic payloads: a stable, non-integrating, self-replicating and biocompatible intracellular platform that ensures controlled expression. Synthetic chromosomes provide the breakthrough in biological bandwidth required to manage large genetic payload delivery and a genetic focal point by which formerly unlinked phenotypic traits can be brought together in a stable, non-integrating, self-replicating, and portable bioengineering system. The present disclosure provides synthetic chromosomes comprising multiple, regulatable expression cassettes, representing a significant breakthrough in cellular therapeutic technologies and providing the ability to coordinately control and manage expression of large genetic payloads and complex polygenic systems.

As described herein, synthetic chromosomes provide a chromosome-vector based bioengineering system that can be readily purified from host (engineering) cells and transferred to recipient (patient) cells by standard transfection protocols. Further provided is the ability to turn off gene expression once therapy is completed and the expression of gene products from the synthetic chromosome is no longer necessary for the patient. An off switch or an inactivation switch may be used if there is an adverse reaction to the expression of the gene products from the synthetic chromosome requiring termination of treatment. For example, a whole-chromosome-inactivation switch may be used, such that expression of genes on the synthetic chromosome are inactivated but the chromosome-containing cells remain alive. Alternatively, a synthetic chromosome-bearing therapeutic cell-off switch could be used in a cell-based treatment wherein, if the synthetic chromosome is contained within a specific type of cell and the cells transform into an undesired cell type or migrate to an undesirable location and/or the expression of the factors on the synthetic chromosome is deleterious, the switch can be used to kill the cells containing the synthetic chromosome, specifically.

Chromosome inactivation mechanisms have evolved in nature, to compensate for gene dosage in species in which the sexes have different complements of a sex chromosome. In humans, the homogametic sex is female containing two copies of the X chromosome, whereas the heterogametic sex is male and contains only one copy of an X chromosome in addition to one copy of a Y chromosome. A means to inactivate one X chromosome evolved to ensure that males and females have similar expression of genes from the X chromosome. Inactivation is achieved by expression of a long non-coding RNA called Xist (X-inactive specific transcript) that is essential for initiation of X chromosome inactivation but is dispensable for maintenance of the inactive state of the X chromosome in differentiated cells. Xist acts in cis to induce heterchromatization of the chromosome from which it is expressed. The Xist gene is located within a region on the X chromosome called the X inactivation center (Xic) that spans over 1 megabase of DNA and contains both long non-coding RNAs and protein coding genes necessary and sufficient for initiation of X chromosome inactivation. Xist expression is regulated in part by Tsix, which is transcribed antisense across Xist. Expression of Tsix prevents expression of Xist on the active chromosome and deletion of Tsix leads to skewed X inactivation such that the mutated chromosome is always inactivated. Inactivation occurs whenever there is more than one Xic present in a cell; thus, inactivation of the synthetic chromosome incorporating an Xic or specific Xic gene products would occur regardless of the sex of the cell into which it is introduced. Notably, evidence indicates that Xist-induced silencing also can occur on autosomes. The Xist cDNA has been inducibly expressed on one chromosome 21 in trisomy 21-induced pluripotent stem cells and demonstrated to induce heterochromatization and silencing of that chromosome 21. (See, e.g., Jiang J, et al., Nature 500 (7462): 296-300 (2013)). Because Xic contains all the cis acting elements necessary for Xist expression and subsequent chromosome inactivation, Xic more accurately recapitulates natural silencing. Pluripotency factors expressed in stem cells and induced pluripotent stem cells (iPSCs) prevent Xist expression; therefore, expression of a therapeutic from a synthetic chromosome incorporating Xic would occur in stem cells and be silenced through chromosome inactivation as the cells become differentiated. Thus, embodiments of the invention contemplate inclusion on a synthetic chromosome of an entire Xic region, or inclusion of select regions, including Xist with or without Tsix.

In some embodiments, one or more regulatory switches may be included as 1) whole-chromosome inactivating switches (comprising an X chromosome inactivation center (Xic) taken from an X chromosome, and/or specific gene sequences from the Xic, including Xist with or without Tsix) and/or 2) gene expression cassette regulatory switches that do not inactivate the whole synthetic chromosome, but instead regulate expression of one or more individual genes on the synthetic chromosome.

In some embodiments, an independent safety switch based on X-chromosome inactivation is employed, in which expression of an X-inactivation specific transcript (Xist) lncRNA results in inactivation of the hSynC chromosome. In some embodiments, the synthetic chromosome comprises an entire Xic region from an X chromosome, and in other embodiments, the synthetic chromosome comprises select sequences from the Xic region of the X chromosome, including the Xist locus, and in some embodiments, further comprising a Tsix locus.

In some embodiments, a regulatory RNA (e.g., an inhibitory RNA) may be produced by induction of the promoter. In some embodiments, a regulatory RNA may be used to regulate an endogenous gene product, or a promoter or a transcript produced by the synthetic chromosome.

As used herein, the term “Xic” refers to sequences at the X inactivation center present on the X chromosome that control the silencing of that X chromosome. As used herein, the term “Xist” refers to the X-inactive specific transcript gene that encodes a large non-coding RNA that is responsible for mediating silencing of the X chromosome from which it is transcribed. “Xist” refers to the RNA transcript. As used herein, the term “Tsix” refers to a gene that encodes a large RNA which is not believed to encode a protein. “Tsix” refers to the Tsix RNA, which is transcribed antisense to Xist; that is, the Tsix gene overlaps the Xist gene and is transcribed on the opposite strand of DNA from the Xist gene. Tsix is a negative regulator of Xist. As used herein, the term “Xic” also refers to genes and nucleic acid sequences derived from nonhuman species and human gene variants with homology to the sequences at the X inactivation center present on the X chromosome that control the silencing of that X chromosome in humans.

In some embodiments, the Xic or select Xic gene product expression cassette is inserted into a synthetic chromosome to provide transcriptional and translational regulatory sequences, and in some embodiments provides for inducible or repressible expression of Xic gene products. In general, the transcriptional and translational regulatory sequences may include, but are not limited to, promoter sequences, ribosomal binding sites, transcriptional start and stop sequences, translational start and stop sequences, repressible sequences, and enhancer or activator sequences.

In general, the regulatable (inducible/repressible) promoters of use in the present invention are not limited, as long as the promoter is capable of inducing (i.e., “turning on” or “upregulating”) or repressing (i.e., “turning off” or “downregulating”) expression of the downstream gene in response to an external stimulus. One such system involves tetracycline-controlled transcriptional activation where transcription is reversibly turned on (Tet-On) or off (Tet-Off) in the presence of the antibiotic tetracycline or a derivative thereof, such as doxycycline. In a Tet-Off system, expression of tetracycline response element-controlled genes can be repressed by tetracycline and its derivatives. Tetracycline binds the tetracycline transactivator protein, rendering it incapable of binding to the tetracycline response element sequences, preventing transactivation of tetracycline response element-controlled genes. In a Tet-On system on the other hand, the tetracycline transactivator protein is capable of initiating expression only if bound by tetracycline; thus, introduction of tetracycline or doxycycline initiates the transcription of the Xic gene product in toto or specific Xic genes. Another inducible promoter system known in the art is the estrogen receptor conditional gene expression system. Compared to the Tet system, the estrogen receptor system is not as tightly controlled; however, because the Tet system depends on transcription and subsequent translation of a target gene, the Tet system is not as fast-acting as the estrogen receptor system. Alternatively, a Cumate Switch Inducible expression system—in the repressor configuration—may be employed. The Cumate Switch Inducible expression system is based on the bacterial repressor controlling the degradative pathway for p-cymene in Pseudomonas putida. High levels of the reaction product, p-cumate, allow binding of the repressor CymR to the operator sequences (CmO) of the p-cym and p-cmt operon. Other regulatable (inducible/repressible) systems employing small molecules are also envisioned as useful in the methods and compositions of the present disclosure.

The entire Xic region may be loaded on to the synthetic chromosome due to the ability of synthetic chromosomes to accommodate very large genetic payloads (>100 Kilo basepairs and up to Megabasepairs (Mbps) in length), or select regions from Xic may be used, including Xist with or without Tsix. The Tsix-Xist genomic region is located on the long arm of the X chromosome at Xq13.2. The Xist and Tsix long non-coding RNAs are transcribed in antisense directions. The Xist gene is over 32 Kb in length while the Tsix gene is over 37 Kb in length. In addition, the entire X chromosome inactivation center, Xic (>1 Mbp in size), may be loaded onto the synthetic chromosome, e.g., as a series of overlapping, engineered BACs.

Certain animal models of diseases are useful in embodiments of the instant disclosure; for example, there are murine and dog models available such as Dys⁻, Utrp⁻, animal models and cell lines.

For example, a “microminipig” model for DMD, made by co-injecting embryos with Cas9 protein and a single-guide RNA targeting exon 23 of the DMD gene for precise editing, was recently reported (Otake, M., et al., Communications Biology, 7 (1): 523; DOI: 10.1038/s42003-024-06222-5). These DMD-edited microminipigs exhibited pronounced clinical phenotypes, including perturbed locomotion and body-wide skeletal muscle weakness and atrophy at one month of age (m.o.a.), respiratory and cardiac dysfunctions by six m.o.a., and a maximum lifespan of 29.9 months, alongside augmented serum creatine kinase levels. Histopathological evaluations confirmed dystrophin deficiency and pronounced dystrophic pathology in the skeletal and myocardial tissues.

The correction of a genetic deficiency in iPS cells derived from mdx mice (a DMD model) and a human DMD patient using a HAC with a complete genomic dystrophin sequence (DYS-HAC) was reported by Kazuki, et al., (2010, Molecular Therapy 18 (2): 386-393).

Satellite cells provide a muscle stem cell niche that may be particularly useful in the treatment of muscular dystrophies. The self-renewing proliferation of satellite cells not only maintains the stem cell population but also provides numerous myogenic cells, which proliferate, differentiate, fuse, and lead to new myofiber formation and reconstitution of a functional contractile apparatus. The complex behavior of satellite cells during skeletal muscle regeneration is tightly regulated through the dynamic interplay between intrinsic factors within satellite cells and extrinsic factors constituting the muscle stem cell niche/microenvironment. (Yin, et al., Physiol. Rev. 2013, 93 (1): 23-67).

Other kinds of stem cells useful in some embodiments of the present disclosure are iPSCs, NSCs, MSCs, MABs, human umbilical huMSCs, adipose-derived, aorta-derived MABs, HIDEMs. For review, see Judson, R. N. and Rossi, F. M. V. (Regen. Med., 2020, 5 (10): 1-6).

The synthetic chromosome systems described herein are also useful for the generation of transgenic animals and ex vivo therapy modeling. (Lindenbaum M, Perkins E, Csonka E, et al. Nucleic Acids Res. 2004, 32 (21): e172; Katona R L, Sinkó I, Holló G, et al. Cell. Mol. Life Sci. 2008, 65 (23): 3830-3838; Monteith D P, Leung J D, Borowski A H, et al. Methods Mol. Biol. 2004, 240:227-242).

Other cells can include myogenic cells, smooth muscle cells, cardiac muscle cells, skeletal muscle cells, syncytia comprising multiple fused cells, sarcomeres, myotubes, muscle fibers, progenitor cells, satellite cells.

Extensive research on satellite cells and their niche has elucidated many cellular and molecular mechanisms underlying skeletal muscle regeneration. Such studies can further the development of therapeutic strategies and serve to alleviate the physiological and pathological conditions associated with poor muscle regeneration observed in sarcopenia and muscular dystrophy. (Yin, H., et al., Physiol. Rev., 2013, 93 (1): 23-67).

A skilled artisan will understand many genetic and biochemical pathways, enzyme mechanisms, signaling pathways, inflammatory and immune responses that can occur in cells, tissues, or a subject/patient, and such current knowledge influence the choices made in designing the presently disclosed SynC compositions, cellular therapeutics and developing the method described herein, yet such knowledge may not be explicitly set forth herein. Nonetheless, such knowledge available to skilled artisans can include the understanding of various signaling pathways directly (e.g. TLRs and NLRP3 inflammasome) in innate immune cells (e.g. DCs, monocytes, macrophages, and neutrophils). Subsequent activation of NFκB and other transcription factors (e.g. IRF3) leads to production and secretion of soluble cytokines, chemokines, and interferons as well as ensuing adaptive immune responses. Furthermore, it may be of interest in the presently disclosed compositions and methods to modulate both innate and adaptive immune cells simultaneously to achieve treatment advantages over only one system of immune response in a subject.

Exemplified herein is a cell-based therapy for treating Duchenne muscular dystrophy (DMD), the most severe form of muscular dystrophy, which results in muscle degeneration and premature death. DMD is an X-linked single-gene defect resulting in a lack of functional dystrophin protein. The dystrophin gene is believed to be the largest gene in the human genome, having a genomic locus (Xp21.2-p21.1) of more than 2-million base pairs (2 Mbps). The dystrophin gene has 79 exons, encoding a 427 kDa protein. DMD is fatal and degenerative, and is estimated to affect 1 in 5000 newborn males worldwide. Symptoms usually first appear between the ages of three and five years, and by age 12, many patients will need wheelchair assistance.

As a component of the dystrophin-associated protein complex (DAPC), the dystrophin protein provides structural support for muscle cell sarcolemma through the attachment of specific regions to anchor points, including the intracellular actin cytoskeleton, the extracellular matrix via the DAPC, and the sarcolemma itself. The absence of functional dystrophin protein leads to loss of sarcolemma structural support, failure of DAPC assembly, and consequent decrease in sarcolemma integrity. This results in continual contraction-induced injury to muscle tissues, which ultimately exhausts the components necessary for myofiber repair and regeneration, resulting in irreversible loss of muscle function. This impacts all skeletal muscles including the diaphragm as well as cardiac muscle and heart function. Individuals with muscular dystrophies lack or produce insufficient dystrophin, leading to muscle cell damage and replacement with fat and fibrotic tissue over time. For protein structure models of full-length dystrophin protein and examples of synthetic mini- and micro-dystrophin constructs, see Asher, et al., (Expert Opin. Biol. Ther., 2020, 20 (3): 263-274).

hSynC allows us to encode multiple genes to make entire protein complexes (˜DAPC), add multiple genes for cellular facilitating factors (to influence, augment or enhance survival, proliferation of the SCs in vitro, or induce, enhance, activate or repress gene expression). The mSynC/hSynC also allows enhancement of target muscle cells, cell homing can be added, and cells carrying the mSynC can be sorted and cells introduced into a tissue or a patient can be tracked.

Congenital muscle diseases, e.g., Duchenne Muscular Dystrophy (DMD) or Becker Muscular Dystrophy (BMD).

### DMD and Other Dystrophies

DMD and LGMD are the most common forms of muscular dystrophies. DMD results from mutations in the dystrophin gene. LGMD results from mutations in up to 31 different loci in the genome, including genes in calpain 3, dysferlin, α-, β-, δ-, or γ-sarcoglycan, desmin, caveolin 3, and many others. (See Berry, S. E., Stem Cells Transl. Med., 2015 January; 4 (1): 91-98).

Some clinical studies of single gene replacement therapy for neuromuscular disorders have shown they can slow or stop disease progression, but such therapies have had little impact on reversing muscle disease that was already present. Promising therapies should reverse disease in patients with muscular dystrophy, and new muscle mass and strength must be rebuilt at the same time that gene replacement prevents subsequent disease. Recently, a dual FKRP/FST gene therapy packaged into a single AAV vector was reported to show promise in normalizing ambulation, increasing strength, decreasing pathology, and showing amplified gene expression in LGMDR9 FKRP_{P448L} mice (Lam, et al., Molecular Therapy, Aug. 7, 2024, 32 (8): 2604-2623).

The presently disclosed innovative technology utilizes a non-viral vector system that has the capacity to carry a full length and fully functional dystrophin gene plus additional nucleic acid sequences (even multiple additional very large sequences) with intentionally designed stoichiometric ratios of genes to be coordinately expressed in host cells, and further encoding sophisticated regulatory switches and facilitators of expression, such that the present synthetic chromosome can act as a multigenic biological circuit board for treatment of DMD.

A patient's or subject's autologous cells can be used for autologous intramuscular transplantation.

Other proteins that can be expressed from SynC: Utrophin, Dystrophin; PAX7; GALGT2 (acetylgalactosaminyltransferase upregulates utrophin and dystrophin); nestin; and/or homologs, orthologs or paralogs thereof (See Berry, S. E., Stem Cells Transl. Med., 2015 January; 4 (1): 91-98).

Tables 1 and 2 list examples of Dystrophin gene homologs and orthologs from various species and some exemplary mRNA transcript variants and synthetic constructs, respectively; many more examples are available in public sequence databases.

TABLE 1

| | | | | # | | | |
|---|---|---|---|---|---|---|---|
| Genus | | | | amino | base | | Accession |
| species | Gene ID | Locus | CCDS | acids | pairs | exons | number |
| Homo | 1756 | Chromosome X | 14233 | 3685 | 11058 | 79 | NM_004006.3 |
| sapiens | (isoform | -NC_000023.11; | | | | | |
| | Dp427m) | Xp21.2-p21.1 | | | | | |

www.ncbi.nlm.nih.gov/datasets/gene/id/1756/products

| | | | | | | |
|---|---|---|---|---|---|---|
| Canis lupus | 606758 | Chromosome X | 3698 | 11097 | 86 | XM_038448874.1 |
| familiaris | (isoform | - NC_051843.1 | | | | |
| | X1) | | | | | |

www.ncbi.nlm.nih.gov/nucleotide/XM_038448874.1

| | | | | | | | |
|---|---|---|---|---|---|---|---|
| Mus | 13405 | Chromosome X | 41047 | 3678 | 11037 | 79 | NM_007868.6 |
| musculus | | - NC_000086.7 | | | | | |

www.ncbi.nlm.nih.gov/nucleotide/NM_007868.6

| | | | | | | |
|---|---|---|---|---|---|---|
| Rattus | 24907 | Chromosome X | 3677 | 11034 | 79 | NM_001370876.1 |
| norvegicus | | - NC_086039.1 | | | | |

www.ncbi.nlm.nih.gov/nucleotide/NM_001370876.1

From: DMD orthologs-NCBI (www.ncbi.nlm.nih.gov/gene/1756/ortholog/?scope=7776).

TABLE 2

| | base | Database |
|---|---|---|
| Description | pairs | Accession No. |
| Homo sapiens dystrophin (DMD), | 13992 | NM_004006.3 |
| transcript variant Dp427m, mRNA | | |
| PREDICTED: Homo sapiens dystrophin | 13960 | XM_006724468.3 |
| (DMD), transcript variant X1, mRNA | | |
| Homo sapiens dystrophin (DMD) | 13957 | M18533.1 |
| mRNA, complete cds | | |
| PREDICTED: Homo sapiens dystrophin | 13960 | XM_054326618.1 |
| (DMD), transcript variant X1, mRNA | | |
| Human mRNA for dystrophin | 12446 | X14298.1 |
| Homo sapiens dystrophin (DMD), | 13854 | NM_000109.4 |
| transcript variant Dp427c, mRNA | | |
| Homo sapiens dystrophin (DMD), | 14083 | NM_004010.3 |
| transcript variant Dp427p2, mRNA | | |
| Homo sapiens dystrophin (DMD), | 14000 | NM_004009.3 |
| transcript variant Dp427p1, mRNA | | |
| Homo sapiens dystrophin (DMD), | 4605 | NM_004018.3 |
| transcript variant Dp71ab, mRNA | | |
| PREDICTED: Homo sapiens dystrophin | 13822 | XM_006724469.4 |
| (DMD), transcript variant X2, mRNA | | |
| PREDICTED: Homo sapiens dystrophin | 13822 | XM_054326619.1 |
| (DMD), transcript variant X2, mRNA | | |
| PREDICTED: Homo sapiens dystrophin | 13662 | XM_006724475.3 |
| (DMD), transcript variant X8, mRNA | | |
| PREDICTED: Homo sapiens dystrophin | 13953 | XM_017029328.2 |
| (DMD), transcript variant X4, mRNA | | |
| PREDICTED: Homo sapiens dystrophin | 13630 | XM_006724474.4 |
| (DMD), transcript variant X7, mRNA | | |
| PREDICTED: Homo sapiens dystrophin | 13921 | XM_006724470.4 |
| (DMD), transcript variant X3, mRNA | | |
| PREDICTED: Homo sapiens dystrophin | 13921 | XM_054326620.1 |
| (DMD), transcript variant X3, mRNA | | |
| PREDICTED: Homo sapiens dystrophin | 13953 | XM_054326621.1 |
| (DMD), transcript variant X4, mRNA | | |
| PREDICTED: Homo sapiens dystrophin | 13630 | XM_054326624.1 |
| (DMD), transcript variant X7, mRNA | | |
| PREDICTED: Homo sapiens dystrophin | 13662 | XM_054326625.1 |
| (DMD), transcript variant X8, mRNA | | |
| PREDICTED: Homo sapiens dystrophin | 10595 | XM_011545468.3 |
| (DMD), transcript variant X9, mRNA | | |
| PREDICTED: Homo sapiens dystrophin | 20305 | XM_054326626.1 |
| (DMD), transcript variant X9, mRNA | | |
| Synthetic construct Homo sapiens | 11119 | BC111587.2 |
| clone IMAGE: 40080544, | | |
| MGC: 133407 DMD protein (DMD) | | |
| mRNA, encodes complete protein | | |
| Synthetic construct Homo sapiens | 11116 | BC118002.1 |
| clone IMAGE: 40080832, | | |
| MGC: 133386 DMD protein (DMD) | | |
| mRNA, encodes complete protein | | |
| Synthetic construct Homo sapiens | 11116 | BC111934.1 |
| clone IMAGE: 40080736, | | |
| MGC: 133449 DMD protein (DMD) | | |
| mRNA, encodes complete protein | | |
| Shuttle vector phcAd.DYS-FL, | 30440 | EU048698.1 |
| complete sequence | | |

The hSynC has the ability to encode multiple genes to make entire protein complexes, add multiple genes for cellular facilitating factors (to influence, augment or enhance survival, proliferation of the SCs in vitro, or induce, enhance, activate or repress gene expression). Such “facilitators” can also enhance target specific cells, can allow tracking cell, homing them to target tissue, and can even be used to sort cells carrying the mSynC.

With such a large carrying capacity, the presently disclosed mSynC can encode antibody scFvs, as well as CARs.

The synthetic chromosomes described herein can be used to engineer stem cells (Vanderbyl S, MacDonald GN, Sidhu S, et al. Stem Cells. 2004, 22 (3): 324-333; Vanderbyl S L, Sullenbarger B, White N, et al. Exp. Hematol. 2005, 33 (12): 1470-1476; Monteith D P, Leung J D, Borowski A H, et al. Methods Mol. Biol. 2004, 240:227-242).

ON/OFF regulatory switches. Safety switches that can turn off SynC or eliminate SynC-carrying cells from tissue or body entirely. In some instances, safety switches can include “ON” components that act as expression accelerators and “OFF” components that decelerate expression/serve a braking function. In some embodiments, the safety switches are one-way switches (e.g., regulating expression from the SynC from OFF to ON, and once the switch changes to “ON,” the SynC-carrying cells enter an irreversible course toward elimination from a tissue and/or patient).

### Cell Lines

Target cells can be primary-culture cell lines established for the purpose of synthetic chromosome production specific for an individual. Alternatively, in some embodiments, the cells to be engineered and/or produce the synthetic chromosome are from an established cell line.

Also contemplated are embryonic cell lines; pluripotent cell lines; adult derived stem cells; or broadly embryonic or reprogrammed cell lines. Further contemplated are primary or cultured cell lines from domesticated pet, livestock and/or agriculturally significant animals, such as dogs, cats, rabbits, hares, pikas, cows, sheep, goats, horses, donkeys, mules, pigs, chickens, ducks, fishes, lobsters, shrimp, crayfish, eels, or any other food source animal or plant cell line of any species. Specifically contemplated are avian, bovine, canine, feline, porcine and rodent (rats, mice, etc.) cells, as well as cells from any ungulate, e.g., sheep, deer, camel goat, llama, alpaca, zebra, or donkey. Cell lines from eukaryotic laboratory research model systems, such as Drosophila and zebrafish, are specifically contemplated. Primary cell lines from zebras, camels, dogs, cats, horses, and chickens (e.g., chicken DT40 cells), are specifically contemplated.

Also contemplated are methods of rescuing wildlife or endangered species (polar bears, ringed seals, spider monkeys, tigers, whales, sea otters, sea turtles, bison, for example) at risk of becoming extinct due to factors such as habitat loss (e.g., due to invasion of another species, human development and/or global warming) or poaching. (See the International Union for Conservation of Nature (IUCN) Red List). Species (plant or animal) that may become endangered and may be in need of rescue due to global warming trends are explicitly contemplated. Also contemplated is the use of the presently claimed cell+synthetic chromosome composition to engineer plant cells to become more nutritive, such as engineering crop plant cells to comprise synthetic chromosomes to carry one or more genes (i) enhancing survival of the plant cell, and/or (ii) enhancing its nutritive value when the plant is eaten.

As used herein, the term “stem cells” can refer to embryonic stem cells, fetal stem cells, adult stem cells, amniotic stem cells, induced pluripotent stem cells (“iPS cells” or “iPSCs”), or any cell with some capacity for differentiation and/or self-renewal. iPS cells are adult cells reprogrammed to exhibit pluripotent capabilities.

As used herein, the term “adult-derived mesenchymal stem cells” (“MSCs”) refers to cells that can be isolated from bone marrow, adipose tissue, peripheral blood, dental pulp, lung tissue or heart tissue from a non-fetal animal. Human MSCs are known to positively express cell surface markers CD105 (SH2), CD73 (SH3), CD44 and CD90, and do not express cell surface markers CD45, CD34, CD14, CD11b, or HLA-DR. Adult-derived mesenchymal stem cells exhibit plastic-adherence under standard culture conditions, are able to develop as fibroblast colony forming units, and are competent for in vitro differentiation into osteoblasts, chondroblasts and adipocytes. “hMSCs” as used herein refers to human adult-derived mesenchymal stem cells.

In some embodiments, the preferred cell lines are mammalian. In some embodiments, the cell lines are human. In some embodiments, the cell lines are from domesticated animals or agricultural livestock. In some embodiments, the cell lines are mesenchymal stem cells, including human mesenchymal stem cells (hMSCs). In some embodiments, the cell lines are pluripotent or induced pluripotent stem cells (iPSCs).

In some embodiments, the cells to be engineered and/or produce the synthetic chromosome are from an established cell line. A wide variety of cell lines for tissue culture are known in the art. Examples of cell lines include but are not limited to human cells lines such as 293-T (embryonic kidney), 721 (melanoma), A2780 (ovary), A172 (glioblastoma), A253 (carcinoma), A431 (epithelium), A549 (carcinoma), BCP-1 (lymphoma), BEAS-2B (lung), BR 293 (breast), BxPC3 (pancreatic carcinoma), Cal-27 (tongue), COR-L23 (lung), COV-434 (ovary), CML T1 (leukemia), DUI45 (prostate), DuCaP (prostate), eHAP fully-haploid engineered HEK293/HeLa wild-type cells, FM3 (lymph node), H1299 (lung), H69 (lung), HCA2 (fibroblast), HEK0293 (embryonic kidney), HeLa (cervix), HL-60 (myeloblast), HMEC (epithelium), HT-29 (colon), HT1080 (fibrosarcoma), HUVEC (umbilical vein epithelium), Jurkat (T cell leukemia), JY (lymphoblastoid), K562 (lymphoblastoid), KBM-7 (lymphoblastoid), Ku812 (lymphoblastoid), KCL22 (lymphoblastoid), KGI (lymphoblastoid), KYO1 (lymphoblastoid), LNCap (prostate), Ma-Mel (melanoma), MCF-7 (mammary gland), MDF-10A (mammary gland), MDA-MB-231, -468 and -435 (breast), MG63 (osteosarcoma), MOR/0.2R (lung), MONO-MAC6 (white blood cells), MRC5 (lung), NCI-H69 (lung), NALM-1 (peripheral blood), NW-145 (melanoma), OPCN/OPCT (prostate), Peer (leukemia), Raji (B lymphoma), Saos-2 (osteosarcoma), Sf21 (ovary), Sf9 (ovary), SiHa (cervical cancer), SKBR3 (breast carcinoma), SKOV-2 (ovary carcinoma), T-47D (mammary gland), T84 (lung), U373 (glioblastoma), U87 (glioblastoma), U937 (lymphoma), VCaP (prostate), WM39 (skin), WT-49 (lymphoblastoid), and YAR (B cell). In some embodiments non-human cell lines may be employed. Rodent cell lines of interest include but are not limited to 3T3 (mouse fibroblast), 4T1 (mouse mammary), 9L (rat glioblastoma), A20 (mouse lymphoma), ALC (mouse bone marrow), B16 (mouse melanoma), B35 (rat neuroblastoma), bEnd.3 (mouse brain), C2C12 (mouse myoblast), C6 (rat glioma), CGR8 (mouse embryonic), CT26 (mouse carcinoma), E14Tg2a (mouse embryo), EL4 mouse leukemia), EMT6/AR1 (mouse mammary), Hepalc1c7 (mouse hepatoma), J558L (mouse myeloma), MC-38 (mouse adenocarcinoma), MTD-1A (mouse epithelium), RBL (rat leukemia), RenCa (mouse carcinoma), X63 (mouse lymphoma), YAC-1 (mouse Be cell), BHK-1 (hamster kidney), DG44 Chinese Hamster Ovary cell line, and CHO (hamster ovary). Plant cell lines of use include but are not limited to BY-2, Xan-1, GV7, GF11, GT16, TBY-AtRER1B, 3n-3, and G89 (tobacco); VR, VW, and YU-1 (grape); PAR, PAP, and PAW (pokeweed); Spi-WT, Spi-1-1, and Spi12F (spinach); PSB, PSW and PSG (sesame); A.per, A.pas, A.plo (asparagus); Pn and Pb (bamboo); and DG330 (soybean). These cell lines and others are available from a variety of sources known to those with skill in the art (see, e.g., the American Type Culture Collection (ATCC) (Manassas, VA.)). These cell lines and others are available from a variety of sources known to those with skill in the art (see, e.g., the American Type Culture Collection (ATCC) (Manassas, VA.)).

Dendritic cells and NK cells may also be used.

Of particular interest are patient autologous cell lines, allogeneic cells, as well as cell lines from a heterologous patient with a similar condition to be treated. In some embodiments, the HT1080 human cell line is employed.

A cell transfected with one or more vectors described herein is used to establish a new cell line, which may comprise one or more vector-derived sequences. The synthetic chromosome producing cell line can then be maintained in culture, or alternatively, the synthetic chromosome(s) can be isolated from the synthetic chromosome producing cell line and transfected into a different cell line for maintenance before ultimately being transfected into a target cell, such as a mammalian cell.

### Synthetic Chromosome Production

Bioengineering of the mSynC platform initially involves the construction of a gene(s) of interests to be placed onto SynC targeting vectors with appropriate selectable markers utilizing standard molecular biology techniques. Targeting vectors can include standard, single-copy bacterial artificial chromosome vectors for carrying large DNA or genomic inserts to bacterial high-copy vectors for carrying smaller DNA inserts. After construction of the targeting vector carrying the gene(s) of interest, the engineered targeting vector along with a vector expressing the SynC unidirectional integrase are co-transfected by standard transfection methodology into a SynC engineering cell line that carries the platform synthetic chromosome. After an appropriate length of time under drug selection, drug resistant clones are selected and confirmation of loading of the targeting vector onto the platform is validated by PCR using appropriate primer pairs that monitor the formation of correct recombination junctions between the targeting vector and the platform synthetic chromosome. The validated and confirmed clones are expanded and the engineered SynC carrying the gene(s) of interest is isolated and subsequently transferred into target therapeutic cells by standard transfection methods.

The synthetic chromosomes of the present disclosure may be produced by any currently employed methods of synthetic chromosome production. As discussed briefly above, the real-time monitoring methods of the present invention are applicable to all of the “bottom up”, “top down”, engineering of minichromosomes, and induced de novo chromosome generation methods used in the art.

The “bottom up” approach of synthetic chromosome formation relies on cell-mediated de novo chromosome formation following transfection of a permissive cell line with cloned α-satellite sequences, which comprise typical host cell-appropriate centromeres and selectable marker gene(s), with or without telomeric and genomic DNA. (For protocols and a detailed description of these methods see, e.g., Harrington, et al., Nat. Genet., 15:345-55 (1997); Ikeno, et al., Nat. Biotechnol., 16:431-39 (1998); Masumoto, et al., Chromosoma, 107:406-16 (1998), Ebersole, et al., Hum. Mol. Gen., 9:1623-31 (2000); Henning, et al., PNAS USA, 96:592-97 (1999); Grimes, et al., EMBO Rep. 2:910-14 (2001); Mejia, et al., Genomics, 79:297-304 (2002); and Grimes, et al., Mol. Ther., 5:798-805 (2002).) Both synthetic and naturally occurring α-satellite arrays, cloned into yeast artificial chromosomes, bacterial artificial chromosomes, or P1-derived artificial chromosome vectors have been used in the art for de novo synthetic chromosome formation. The products of bottom-up assembly can be linear or circular, comprise simplified and/or concatamerized input DNA with an α-satellite DNA based centromere, and typically range between 1 and 10 Mb in size. Bottom up-derived synthetic chromosomes also are engineered to incorporate nucleic acid sequences that permit site-specific integration of target DNA sequences onto the synthetic chromosome.

The “top down” approach of producing synthetic chromosomes involves sequential rounds of random and/or targeted truncation of pre-existing chromosome arms to result in a pared down synthetic chromosome comprising a centromere, telomeres, and DNA replication origins. (For protocols and a detailed description of these methods see, e.g., Heller, et al., PNAS USA, 93:7125-30 (1996); Saffery, et al., PNAS USA, 98:5705-10 (2001); Choo, Trends Mol. Med., 7:235-37 (2001); Barnett, et al., Nuc. Ac. Res., 21:27-36 (1993); Farr, et al., PNAS USA, 88:7006-10 (1991); and Katoh, et al., Biochem. Biophys. Res. Commun., 321:280-90 (2004).) “Top down” synthetic chromosomes are constructed optimally to be devoid of naturally occurring expressed genes and are engineered to contain DNA sequences that permit site-specific integration of target DNA sequences onto the truncated chromosome, mediated, e.g., by site-specific DNA integrases.

A third method of producing synthetic chromosomes known in the art is engineering of naturally occurring minichromosomes. This production method typically involves irradiation-induced fragmentation of a chromosome containing a neocentromere possessing centromere activity in human cells yet lacking α-satellite DNA sequences and engineered to be devoid of non-essential DNA. (For protocols and a detailed description of these methods see, e.g., Auriche, et al., EMBO Rep. 2:102-07 (2001); Moralli, et al., Cytogenet. Cell Genet., 94:113-20 (2001); and Carine, et al., Somat. Cell Mol. Genet., 15:445-460 (1989).) As with other methods for generating synthetic chromosomes, minichromosomes can be engineered to contain DNA sequences that permit site-specific integration of target DNA sequences.

The fourth approach for production of synthetic chromosomes involves induced de novo chromosome generation by targeted amplification of specific chromosomal segments. This approach involves large-scale amplification of pericentromeric/ribosomal DNA regions situated on acrocentric chromosomes. The amplification is triggered by co-transfection of excess exogenous DNA specific to the pericentric region of chromosomes, e.g., ribosomal RNA, along with DNA sequences that allow for site-specific integration of target DNA sequences and also a selectable marker, which integrates into the pericentric heterochromatic regions of acrocentric chromosomes. (For protocols and a detailed description of these methods see, e.g., Csonka, et al., J. Cell Sci 113:3207-16 (2002); Hadlaczky, et al., Curr. Opin. Mol. Ther., 3:125-32 (2001); and Lindenbaum, M., Perkins, E., et al., Nucleic Acid Research, 32 (21): e172 (2004)). During this process, upon targeting and integration into the pericentric regions of the acrocentric chromosomes, the co-transfected DNA induces large-scale amplification of the short arms of the acrocentric chromosome (rDNA/centromere region), resulting in duplication/activation of centromere sequences, formation of a dicentric chromosome with two active centromeres, and subsequent mitotic events result in cleavage and resolution of the dicentric chromosome, leading to a “break-off” satellite DNA-based synthetic chromosome approximately 40-80 Mb in size comprised largely of satellite repeat sequences with subdomains of co-amplified transfected transgene that may also contain amplified copies of rDNA, as well as multiple site-specific integration sites. The newly-generated synthetic chromosome can be validated by observation of fluorescent chromosome painting or FISH or FlowFISH or CASFISH (Deng et al., PNAS 2015 112 (38): 11870-11875), via markers that have been incorporated, such as an endogenous chromosome tag and a synthetic chromosome tag, which were engineered into the synthetic chromosome production cell line and/or the synthetic chromosome itself, as the synthetic chromosome was being made.

An artificial chromosome expression system (ACE system) has been described previously as a means to introduce large payloads of genetic information into the cell (Lindenbaum, M., Perkins, E., et al., Nucleic Acid Research, 32 (21): e172 (2004); Perkins et al., US Pat. Pub. No. 20060246586; Perkins et al., US Pat. Pub. No. 20030119104; Perkins, et al., US Pat. Pub. No. 20050181506; and Perkins, et al., U.S. Pat. No. 7,521,240; each of these references is explicitly incorporated by reference in its entirety. Synthetic or ACE platform chromosomes are synthetic chromosomes that can be employed in a variety of cell-based protein production, modulation of gene expression or therapeutic applications. During the generation of synthetic platform chromosomes, unique DNA elements/sequences required for integrase mediated site-specific integration of heterologous nucleic acids are incorporated into the synthetic chromosome which allows for engineering of the synthetic chromosome. By design, and because the integrase targeting sequences are amplified during synthetic chromosome production, a large number of site-specific recombination sites are incorporated onto the synthetic chromosome and are available for the multiple loading of the synthetic platform chromosome by delivery vectors containing multiple gene regulatory control systems.

Thus, the ACE System consists of a platform chromosome (ACE chromosome) containing approximately 75 site-specific, recombination acceptor sites that can carry single or multiple copies of genes of interest using specially designed ACE targeting vectors (pAPP) and a site-specific integrase (ACE Integrase). The ACE Integrase is a derivative of the bacteriophage lambda integrase (INT) engineered to direct site-specific unidirectional recombination in mammalian cells in lieu of bacterial encoded, host integration accessory factors (λINTR). Use of a unidirectional integrase allows for multiple and/or repeated integration events using the same, recombination system without risking reversal (i.e., pop-out) of previous integration/insertions of bioengineered expression cassettes. The transfer of an ACE chromosome carrying multiple copies of a red fluorescent protein reporter gene into human MSCs has been demonstrated (Lindenbaum, M., Perkins, E., et al., Nucleic Acid Research, 32 (21): e172 (2004)). Fluorescent in situ hybridization and fluorescent microscopy demonstrated that the ACEs were stably maintained as single chromosomes and expression of transgenes in both MSCs and differentiated cell types is maintained (Vanderbyl et al., 2004, Stem Cells, 22:324-333).

### Chromosome Transfer

Adipose-derived MSCs can be obtained from Lonza and cultured as recommended by the manufacturer, in which the cells are cultured under a physiological oxygen environment (e.g., 3% O2). A low oxygen culture condition more closely recapitulates the in vivo environment and has been demonstrated to extend the lifespan and functionality of MSCs. Engineered platform chromosomes can be purified away from the endogenous chromosomes of the synthetic chromosome production cells by high-speed, flow cytometry and chromosome sorting, for example, and then delivered into MSCs by commercially available lipid-based transfection reagents. Delivery of intact, engineered ACE platform chromosomes can be confirmed by FISH, Flow-FISH, CASFISH and/or PCR analysis.

Functional Elements and Facilitators May Be Integrated into the Synthetic Chromosome:

Multiple genes in a biochemical pathway may be encoded on the mSynC for expression in a coordinated manner; these may be considered “cellular enhancements,” as they may enhance certain qualities of the cells carrying the synthetic chromosome, such as growth, vitality, longevity, replication ability, etc., lending to an improved method of cellular gene therapy overall. Additional cellular enhancements can include those that augment, elevate or lower a normal cell function and/or cellular activity and/or add a novel cell function (e.g., increased oxygen carrying capacity of blood cells or addition of radioprotective elements such as for astronauts during space travel).

In some embodiments, biological beings (be they animals, plants or microbes) are subjected to an altered environment in space, including microgravity, increased radiation and partial vacuum. Genetic engineering to improve survival or function of the organism in space will require a genetic platform with large carrying capacity amenable to complex payloads. The SynC's capacity, flexibility and portability make it suitable for engineering cyto- and geno-protectants that enhance physiological responses to space, including (but not limited to):
1. Antioxidants Proteins (e.g., superoxide dismutase (SOD), catalase (CAT), glutathione peroxidase (GP), thioredoxin reductase (TSNRD), and Heme oxygenase-1 (HO-1), nuclear factor erythroid 2 (Nrf2));
2. DNA Repair Proteins and Cell Cycle Checkpoint Regulators (e.g., TP53, CDKN1A (p21));
3. Factors for muscle and bone health (e.g., Myostatin inhibitors, insulin-like growth factor 1 (IGF-1), bone morphogenetic proteins (BMPs), heat shock proteins (HSP));
4. Factor for cardiovascular health (e.g., Vascular endothelial growth factor (VEGF), endothelial nitric oxide synthase (eNOS));
5. Factors for immune and blood cell function modulation (e.g., Cytokines (IL-7, IL-15), granulocyte-colony stimulating factor (G-CSF), erythropoietin (EPO)); and
6. Factor for adapting to microgravity (e.g., Small ubiquitin-like modifiers (SUMO), apolipoprotein A-1 (ApoA-1), growth factors).

Delivery of multiple cyto- and genoprotectants potentially fortifies cells from environmental genetic insults such as ionizing radiation, free radical exposure, and other physiological perturbations due to exposure to the microgravity environment.

The use of a synthetic chromosome able to carry extremely large inserts allows for the expression of multiple expression cassettes comprising large genomic sequences, and multiple genes.

A uniquely valuable use of the presently disclosed mammalian synthetic chromosome is that this system can encode and express multiple genes in the most optimal dosages and at fixed stoichiometries of expressed gene products.

In some cases, the word “facilitator” can encompass various regulatory elements (e.g., safety switches on the SynC. In some cases, the word “facilitator” can encompass (but is not limited to) functions added to the SynC to aid cellular delivery, cell targeting to specific tissues, genes encoding growth and/or survival factors, cell longevity factors, and regulators of replication of the SynC-carrying cells. Other cellular enhancements can include those that augment, elevate or lower a normal cell function and/or cellular activity and/or add a novel cell function (e.g., increased oxygen carrying capacity of blood cells or addition of radioprotective elements (space travel)).

In vitro propagation of cell lines may be used in the production of synthetic growth factors, for example.

In vitro propagation of cell lines may be aided by the use of biomaterial like gels, recreating biophysical characteristics similar to in vivo.

Because the hSynC therapy is enduring, any second therapy provided at any point following the hSynC therapy may be considered “in combination.”

At least some forms of current treatments of diseases such as muscular dystrophies involve the use of steroidal, non-steroidal and cytokine-based anti-inflammatory agents, as well as glucocorticoids and follistatin. Controlling fibrosis (block ECM proteins), inflammation, and atrophy (expression of miRNA-29 family), drug: andrographolide, HCT1026, nitric oxide.

Additional therapeutic agents may be encoded on the SynC or may be supplied by a source external to the cell, tissue or the patient's body. The mSynC can be supplied in combination with additional therapeutic agents and/or pharmaceuticals, (e.g., glucocorticoids. follistatin (inhibitor of myostatin) to increase muscle mass and strength). Various methods are being studied in attempts to increase muscle cell growth and regenerative ability: Antisense and RNAi, increasing Dystrophin, exon-skipping, introducing miniDys or microDys, drug Dantrolene, knocking out myostatin (See Cordova, G. et al., Frontiers in Genetics, 2018 April, vol. 9, article 14).

### EXAMPLES

### Example 1

The feasibility of engineering large and multiple genomic fragments onto a generated mouse synthetic chromosome has been demonstrated for human therapeutic applications (Greene et al., MTMCD (2019) 13:463-473). In this Example, the synthetic chromosome-based genetic platform, termed “hSynC,” was engineered to carry the full-length cDNA corresponding to the human dystrophin gene (DMD) (SEQ ID NO: 3). Placement of the DMD cDNA on the hSynC via a unidirectional, site-specific recombination event was catalyzed by a modified phage lambda integrase. Confirmation of the placement of the full-length DMD cDNA on the hSynC was demonstrated by PCR-based assay of recombination junctions as well as fluorescent in situ hybridization. Expression of the DMD cDNA was confirmed by RT-PCR.

The results presented herein demonstrate the efficient and tractable engineering of a human-derived hSynC platform to express both a large human dystrophin cDNA as well as a transcript variant of the DMD gene, Dp71ab (described in Example 2). As a further step in translating the hSynC platform technology for human therapeutic applications, the presently disclosed hSynC system incorporates a genetic safety switch capable of curbing potential side effects.

Construction of DMD cDNA Clones

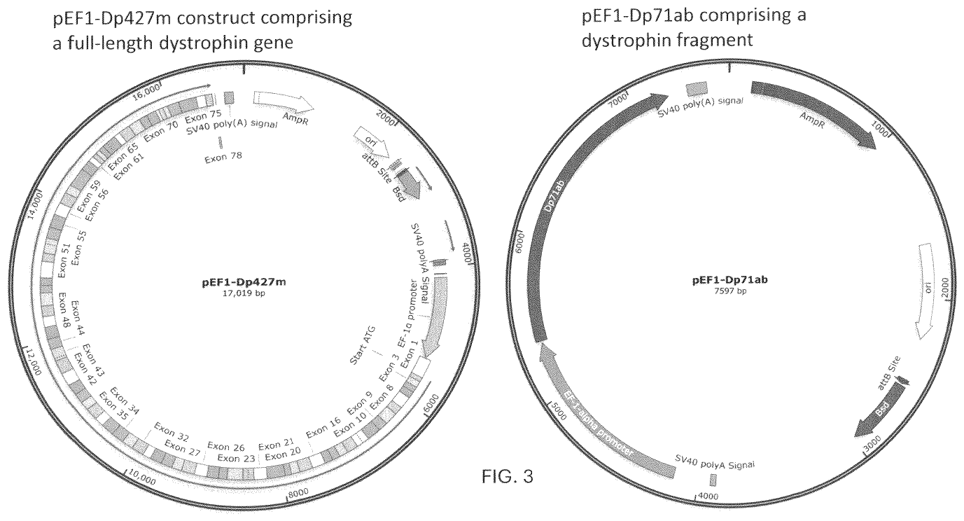

FIG. 3 shows a full-length dystrophin was created by a combination of direct DNA synthesis and PCR amplification of exons 1-79 and cloned by recombination (InFusion Cloning, TaKaRa Bio USA, Inc.) to generate pEF1-Dp427m. The sequence-verified construct was loaded onto the hSynC platform as previously described (Lindenbaum M, Perkins E, Csonka E, et al. Nucleic Acids Res. 2004, 32 (21): e172).

Cloning and Construction of Full-Length Human DMD cDNA hSynC Loading Vector

For isolation of a full-length human DMD cDNA clone encompassing exons 1-79 (GenBank Sequence ID NM_004006.3 is 13992 base pairs in length and comprises the DMD gene coding sequence from bp 238 through 11295), one microgram of human skeletal muscle total RNA (TaKaRa Human Skeletal Muscle Total RNA (Cat #636534; Lot #2211060; normal human skeletal muscle from 4 males/females Asian/Caucasia; ages 27-86) was reversed transcribed with Maxima H Minus Reverse Transcriptase using DMD gene specific primer CGB0676 (Seq #1). One microgram of human skeletal muscle total RNA was added to a solution containing 2 pmol of primer CGB0676, 0.5 mM final concentration of dNTPs for a final solution volume of 15 microliters. The solution was incubated at 65° C. for 5 minutes then chilled on ice. Next, 4 microliters of 5× Maxima RT Buffer was added along with 1 microliter of Maxima H Minus Enzyme Mix for a final volume of 20 microliters. The solution was subsequently incubated for 60 minutes at 50° C. followed by termination of the reaction by incubating at 85° C. for 5 minutes. The reaction was stored at −20° C. prior to second strand cDNA synthesis.

Prior to second strand cDNA synthesis, 2 microliters of the first strand reaction was diluted 1:5 in RNAse-free, DNase-free dH₂O to a final volume of 10 microliters. Eight microliters of the diluted first strand cDNA synthesis reaction was added to a reaction containing 1× TaKaRa Ex Premier DNA Polymerase Mix (TaKaRaBio, Inc., Cat #RR370A), 0.25 micromolar primer CGB0674, 0.25 micromolar primer CGB0635, and RNAse-free, DNase-free dH₂O to a final volume of 200 microliters. This solution was divided into 8×25 microliter reaction tubes and two-step PCR was performed as follows:
7. 94° C. for 1 minute
8. 98° C. for 10 seconds
9. 68° C. for 12 minutes
10. Repeat steps 2 through 3 for a total of 30 cycles
11. 68° C. for 5 minutes
12. 4° C. Hold
13. End

After the completion of second strand cDNA synthesis, all reaction tubes were analyzed by agarose gel electrophoresis using 1×TAE Buffer. Subsequently, the gel was incubated in 1× Sybr Green nucleic acid stain (ThermoFisher, Inc., Cat #S7563) in 1×TAE buffer for 30 minutes at room temperature. The stained gel was visualized and the corresponding 11.3 Kb PCR product of each of the eight samples was isolated from agarose using the NEB Monarch Gel Extraction Kit (NEB, Inc., Cat #T1020S). During the purification process, the eight samples were pooled into a final volume of 19 microliters. At this point, the PCR product ends were converted to blunt-ended, phosphorylated DNA fragments using the NEB Quick Blunting Kit (NEB, Inc., Cat #E1201S). For this, nineteen microliters of the gel purified PCR product was added to 2.5 microliters of 10× Blunting Buffer, 2.5 microliters 1 mM dNTP mix, and 1 microliter of Blunt Enzyme Mix to a final volume of 25 microliters. The solution was incubated at 25° C. for 30 minutes followed by enzyme inactivation at 70° C. for 10 minutes. This reaction product was stored at −20° C. until it was used for subsequent ligation.

The 11.3 Kb DMD cDNA PCR blunt-ended fragment was cloned into the vector SPB0487. Initially, SPB0487 was digested with BamHI/NotI and the vector backbone was blunt-ended with the NEB Quick Blunting Kit. The vector was purified from the Quick Blunting reaction using the NEB Monarch PCR & DNA Cleanup Kit (NEB, Inc, Cat #T1030S) with final column elution of 17 microliters. Subsequently, the purified SPB0487 was dephosphorylated in a 20 microliter reaction solution containing 2 microliters of 10× Antarctic Phosphatase Reaction Buffer and 1 microliter (5 Units) Antarctica Phosphatase (NEB, Inc., Cat #M0289S). This solution was incubated for 30 minutes at 37° C. and the phosphatase was subsequently inactivated by incubating at 80° C. for 2 minutes.

For ligation of the 11.3 Kb DMD cDNA product to SPB0487, equal molar concentrations of each were added to a solution containing 1×T4 DNA ligase buffer and 1 microliter T4 DNA ligase (NEB, Inc., M0202S) and incubated overnight at 16° C. After overnight incubation, 1 microliter of the ligation reaction was transformed into NEB Stable cells with transformants selected overnight after growth at 30° C. on LB+Amp (100 micrograms/ml) agar plates. Confirmation of clones containing the 11.3 Kb fragment in the proper orientation with respect to the human EF1α promoter were confirmed by colony PCR followed by restriction digestion. Correct clones were loaded onto the hSynC as previously described (Greene et al., 2019, Mol. Ther. Methods Clin. Dev., 13:463-473).

The primers used to identify and clone the full-length transcript of dystrophin are listed below as SEQ ID NO: 1 and SEQ ID NO: 2, below.

DMD Primer List

| | | SEQ |
|---|---|---|
| Primer | Sequence | ID: |
| CGB0674 | AGGTGTCGTGACCCGGGATCATCAGTTACTGTGTTGA | 1 |
| | CTCACTCAGT | |
| CGB0635 | TATGATCTAGAGTCGCGGCCACTTCCTACATTGTGTC | 2 |
| | CTCTCTCATTG | |

The sequence of CGB0674 shown in bold matches the 5′end of the full-length coding sequence of the DMD (Dp427m) gene (represented by the nucleotide sequence identified in SEQ ID NO: 3 in the Sequence Listing) contained within the construct loaded onto the hSynC, and the sequence of CGB0635 shown in bold matches the reverse complement of a sequence at 3′ end of SEQ ID NO: 3.

Additional primers were used to sequence the 11.3 Kb DMD gene after cloning, and the full-length coding sequence of DMD (Dp427m) (set forth as SEQ ID NO: 3) was confirmed as the sequence loaded onto the hSynC.

SEQ ID NO: 4 presents the nucleic acid sequence of the vector construct SPB0487=pSTV28-XbaI/BstZ17I ori fragment (to provide a low copy origin of replication) cloned into SpeI/SwaI vector backbone of SPB0471.

### Example 2

The DMD gene consists of 79 exons spanning a 14-kb transcript encoding the protein dystrophin Dp427. Alternative promoters scattered throughout the DMD gene produce at least eight tissue- or development-specific isoforms. Dp71 mRNA is characterized by multiple alternative splicings of exons 71, 71-74, and 78 and intron 77, resulting in the expression of more than 10 types of Dp71 splice variants. Independent skipping of exons 71 and 78 yields Dp71a and Dp71b, respectively, whereas simultaneous skipping produces Dp71ab. Dp71 is believed to play important roles in various cellular processes, including water homeostasis, nuclear architecture and cell adhesion, as well as in cell division and survival, and possibly cancer development. One alternative downstream promoter located in intron 62 produces a transcript consisting of a unique exon, G1 and DMD exons 63-79 shared with other dystrophin isoforms. This transcript encodes a nearly 71-kDa protein, dystrophin Dp71, an isoform that is expressed ubiquitously. It has been demonstrated that dystrophin Dp71ab monoclonally expressed in human satellite cells enhanced proliferation of myoblast cells. In addition, over-expression of Dp71ab enhanced myoblast proliferation, suggesting that expression of Dp71ab may generate a high yield of stem cells for DMD treatment. Full-length Dp71 consisting of 18 exons from exons G1 to 79 was amplified by reverse transcription-PCR from total RNA of human satellite cells. The amplified product showed deletion of both exons 71 and 78 in all sequenced clones, indicating monoclonal expression of Dp71ab. Western blotting of the satellite cell lysate showed a band corresponding to over-expressed Dp71ab. Transfection of a plasmid expressing Dp71ab into human myoblasts significantly enhanced cell proliferation when compared to the cells transfected with the mock plasmid. However, transfection of the Dp71 expression plasmid encoding all 18 exons did not enhance myoblast proliferation. From these studies, it was concluded that Dp71ab, but not Dp71, was a molecular enhancer of myoblast proliferation and that transfection with Dp71ab may generate a high yield of stem cells for DMD treatment. (Farea 2020, Scientific Reports Article Number 17123).

Although the splice variants of Dp71 have been implicated in specific functions, the functional characterization of each splice variant has been hampered by the co-expression of isoforms. The presently described hSynC system represents a uniquely useful way to examine the expression of one or more isoforms of dystrophin genes, alone or together in specific stochiometric ratios, and in specific tissues and/or cells of a patient. As just one example, an mSynC comprising both the full-length dystrophin isoform Dp427m and isoform Dp71ab can be engineered and tested for expression of both variants, and further assessed for the ability of this dual expression construct enhances proliferation of myoblasts.

The DMD Dp71ab was chosen as a good target to potentially increase the effectiveness of the DMD therapeutic. The DMD Dp71ab coding sequence was downloaded from UCSC genome browser and NCBI BlastN confirmed the sequence was 100% identical to the desired isoform (Accession Number NM_004018.3)

It has also been reported that dystrophin Dp71 expression is down-regulated during myogenesis. Dp71 expression is present in myoblasts but declines during myogenesis, presumably to avoid interfering with the function of dystrophin, the predominant DMD gene product in differentiated muscle fibers. The transcriptional regulatory mechanisms operating on the developmentally regulated expression of Dp71 were studied during myogenesis of C2C12 cells. Promoter deletion analysis showed that the 224-bp 5′-flanking region, which contains several Sp-binding sites (Sp-A to Sp-D), is responsible for the Dp71 promoter basal activity in myoblasts as well as for down-regulation of the promoter in differentiated cells. Electrophoretic mobility shift and chromatin immunoprecipitation assays indicated that Sp1 and Sp3 transcription factors specifically bind to the Sp-binding sites in the minimal Dp71 promoter region. Sp-A was reported to be the most important binding site for the proximal Dp71 promoter activity, and cotransfection of the promoter construct with Sp1- and Sp3-expressing vectors into Drosophila SL2 cells, which lack endogenous Sp family, confirmed that these proteins activate specifically the minimal Dp71 promoter. Endogenous Sp1 and Sp3 proteins were detected only in myoblasts and not in myotubes, which indicated that the lack of these factors caused down-regulation of the Dp71 promoter activity in differentiated cells. In corroboration, efficient promoter activity was restored in differentiated muscle cells by exogenous expression of Sp1 and Sp3. (de Leon et al., 2005, JBC 280:5290-5299).

Thus, for the present disclosure, as additional evidence of the utility of the hSynC bioengineering approach to DMD therapeutics, a splice variant of the DMD gene, Dp71ab, was synthesized and engineered onto the hSynC platform. SEQ ID NO: 5 represents the nucleic acid sequence of this synthetic construct of the Dp71 isoform loaded onto the hSynC platform chromosome. The minimal endogenous promoter (600 bp upstream of the transcription start site) was added to the 5′ end of the DMD Dp71ab coding sequence, and this entire sequence (2469 bp) was synthesized by TWIST BioSciences and cloned into their medium copy Amp-R vector. This was then used as a template in PrimeStar PCR reactions with CGB683/CGB685 (to amplify Dp71ab with its endogenous promoter) and CGB684/CGB685 (to amplify just the Dp71ab ORF). The PCR products were gel purified and cloned by InFusion into SPB0471 digested with EcoRI/NotI for the endogenous promoter-Dp71ab (Pr600-Dp71ab) or with BamHI/NotI for cloning the Dp71ab ORF downstream of the EF1α promoter.

FIG. 3 shows a schematic of the Dp71ab construct generated by DNA synthesis (Twist Bioscience, Inc.) and cloned by recombination. The sequence-verified construct was loaded onto the hSynC platform as previously described (Lindenbaum M, Perkins E, Csonka E, et al. Nucleic Acids Res. 2004, 32 (21): e172).

The Dp71ab sequence was confirmed by PCR-based assay of recombination junctions as well as fluorescent in situ hybridization, and its expression confirmed by RT-PCR.

DP71ab Primer List

| | | SEQ |
|---|---|---|
| Primer | Sequence | ID: |
| CGB683 | CAAATAAAGATCCACGAATTCTTATTTTAGCAGGAAAA | 6 |
| | CATGTCCCATGAGAC | |
| CGB684 | AGGTGTCGTGACCCGGGATCCCCTGGCATGAGGGAACA | 7 |
| | GC | |
| CGB685 | TATGATCTAGAGTCGCGGCCGCTTATTCTGCTCCTTCT | 8 |
| | TCATCTGTCATGACT | |

Clones were sequence verified prior to loading onto the hSynC.

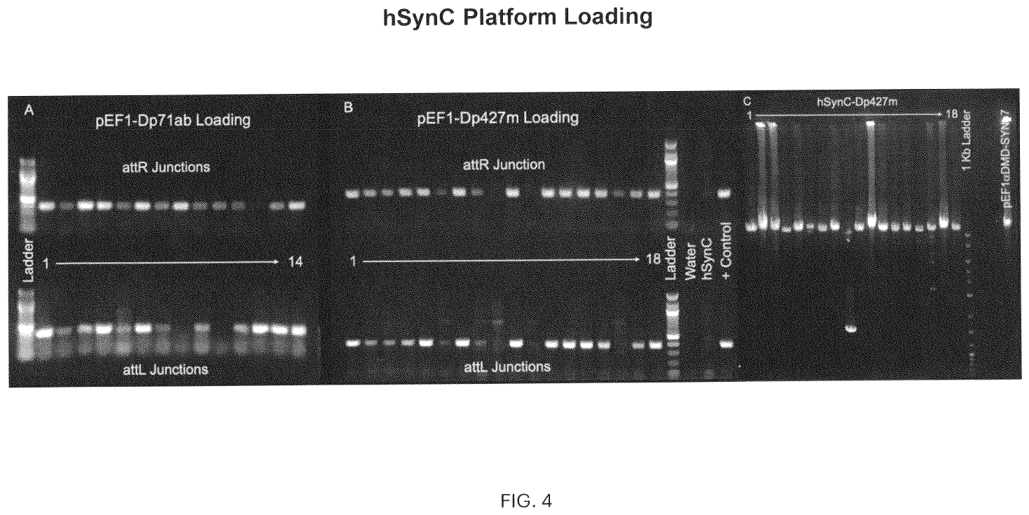

FIG. 4 shows an analysis of hSynC Platform Loading. The hSynC platform cell line CGBc003 (CHO based) was engineered to carry the full-length dystrophin cDNA isoform Dp427m (plasmid pEF1aDMD-SYNp7) or the Dp71ab isoform variant (plasmid pEF1-Dp71ab). Genomic DNA was isolated from individual clones (DNeasy Blood & Tissue Kit with QIAcube Connect; Qiagen, Inc.) and approximately 50 ng of DNA used for PCR detection of the resulting recombination junctions attR and attL using primers that flank the respective recombination junctions. FIG. 4 (A) shows that 11 of 14 clones from the pEF1-Dp71ab loading onto the hSynC were positive for both the attR and attL recombination junction (˜78% success rate). FIG. 4 (B) shows that 15 of 18 clones of the pEF1-Dp427m loading onto the hSynC were positive for both the attR and attL recombination junction (˜83% success rate). To assess the structural integrity of the Dp427m full-length dystrophin variant, purified genomic DNA from the 18 pEF1-Dp427m loaded clones was analyzed by PCR with primers targeting the 5′ and 3′ ends of dystrophin (exons 1 and 79, respectively) using PrimeSTAR HS DNA polymerase reactions conditions according to the manufacturer's protocol (TaKaRa Bio, Inc.). FIG. 4 (C) indicates that 17 of 18 isolated clones contained the intact human DMD cDNA.

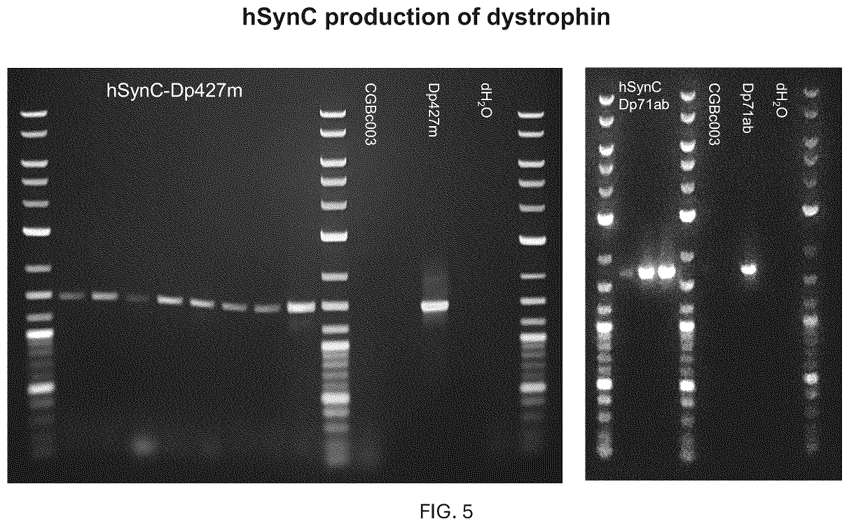

FIG. 5 presents an RNA expression analysis: The hSynC platform cell line CGBc003 was engineered to carry and expresses full-length dystrophin isoform Dp427m (pEF1aDMD-SYNp7) or isoform Dp71ab (pEF1aDp71ab). Total RNA was extracted from isolated clones (RNeasy Mini Kit; Qiagen, Inc.), DNase treated and converted to first-strand cDNA (oligo dT primed; Maxima cDNA Synthesis Kit; Thermo Fisher, Inc.). Primers targeting the 5′ end of Dp427m and the 3′ end of Dp71ab were used to confirm expression of the full-length human DMD gene by PCR (OneTaq; NEB Inc.). All tested clones (eight total for Dp427m and three for DP71ab) exhibit expression of the human DMD gene from the hSynC platform.

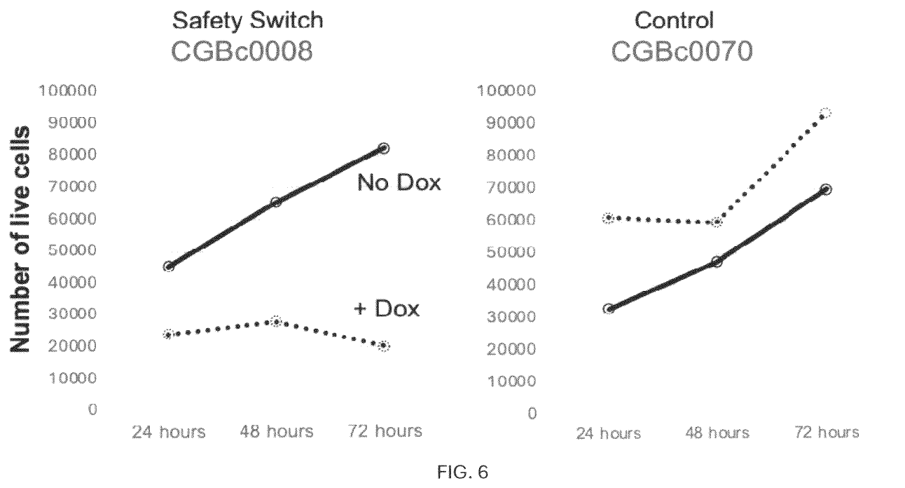

FIG. 6 shows the hSynC targeting vector containing a proapoptotic cassette under the control of a proprietary doxycycline regulatable promoter, loaded onto the platform chromosome (CGBc0008). Induction of the pro-apoptotic signal upon addition of doxycycline to the cell culture media induces a rapid cell death as compared to control cells (CGBc0070). Induction of apoptosis was confirmed by Annexin V staining (data not shown).

Transfer of Confirmed Full-Length DMD Carrying Synthetic Chromosome into HT1080 Cells

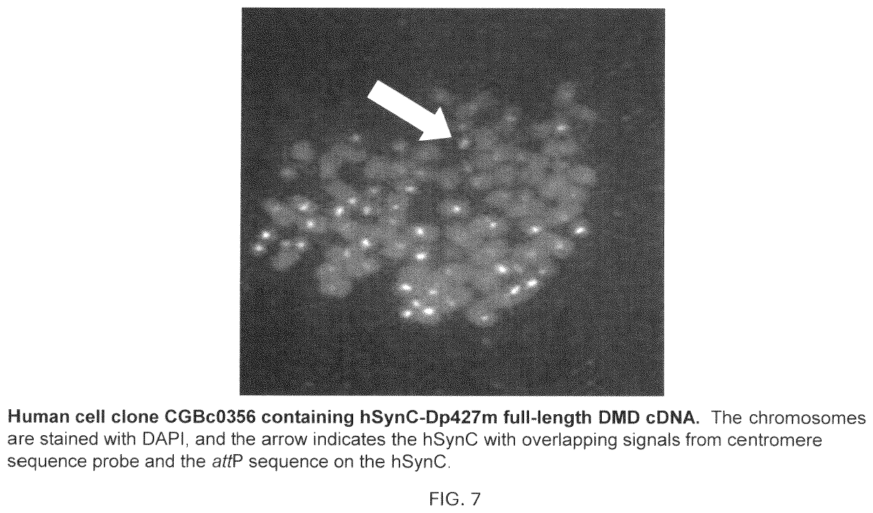

The hSynCs containing either the Dp427m full-length DMD cDNA or the isoform variant Dp71ab were purified from the respective CGBc003 clones and transferred into the luciferase expressing HT1080 clone CGBc0344. The delivery of intact hSynC-Dp427m into HT1080 was confirmed by FISH (FIG. 7).

### Example 3

Confirmation of Full-Length DMD Expression from Human HT1080 Cells Carrying the DMD Gene Expressed from a Synthetic Chromosome

Animal protocol. Two cell lines (CGBc0356 and CGBc0357), each an independent clonal line carrying the synthetic chromosome expressing the full length DMD gene from a human EF1 alpha promoter, were expanded in culture medium (Gibco DMEM (Cat. #11965-092), supplemented with Gibco sodium pyruvate (Cat. #11360-070) at 1 mM, 10% Gibco HI FBS (Cat. #16140-071), 100 U/ml Gibco Penicillin-Streptomycin (500 U/ml, Cat. 15070-063) at 100 U/ml and Gibco Puromycin (10 mg/ml, Cat. #A11138-03). On Study Day 0, each cell line was harvested, washed and resuspended in phosphate buffered saline (PBS) at 50×10⁶ cells/ml. A 1:1 mixture of each cell line with Matrigel was made for a final concentration of 25×10⁶ cells/ml to be implanted into the right flank of subject animals (NOD.Cg-Rag1tm1Mom Il2rgtm1Wjl/SzJ (JAX Stock #007799; NRG). Subject animals were 6-8 weeks of age on Day 0. Five animals were injected with each cell line (HT1080-Nanoluc-EF1a-DMD427m RCA2 and HT1080-Nanoluc-EF1a-DMD427m RC5) and monitored twice weekly for body weight, tumor volume, as well as general health condition under veterinary supervision. On Day 27 or when tumor volumes reach 500-1000 mm³, mice were euthanized and tumors collected. Each tumor was divided into two parts with one half snap frozen and the other half frozen in RNA later.

Validated Expression of DMD Gene from hSynC in Impacted Animals

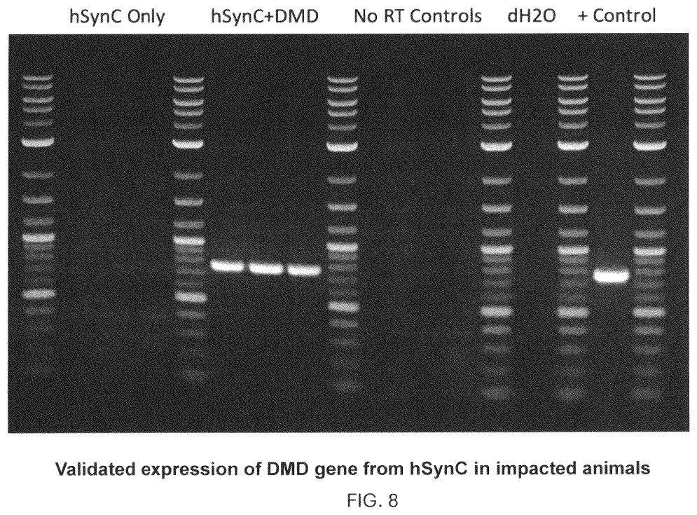

Tissue samples from the injected mice (both cells containing the hSynC+DMD and hSynC only controls) were excised and total RNA from each sample was purified with included DNAase digestion step (RNeasy Mini Kit; Qiagen, Inc.). First strand cDNA synthesis was performed using random hexamers (ThermoFisher Maxima H Minus 1st strand cDNA Synthesis Kit). Subsequent PCR analysis of the first strand synthesis products using primers targeting the human DMD gene were used to confirm expression (FIG. 8).

Our data confirm engineering of the hSynC to express either the full-length DMD cDNA or the isoform Dp71ab. In vitro expression of both hSynC-DMD clones were confirmed by RT-PCR and in vivo expression of the full-length DMD cDNA from the hSynC in a mouse animal model was confirmed by RT-PCR indicating that the hSynC-DMD engineered synthetic chromosome is capable of producing full-length dystrophin both in cells in vitro and in a tissue environment. Experiments are underway to produce the mSynC comprising both the full-length dystrophin isoform Dp427m and isoform Dp71ab, which will be tested for expression of both variants and for whether this mSynC comprising both variants retains the ability to enhance proliferation of myoblasts.

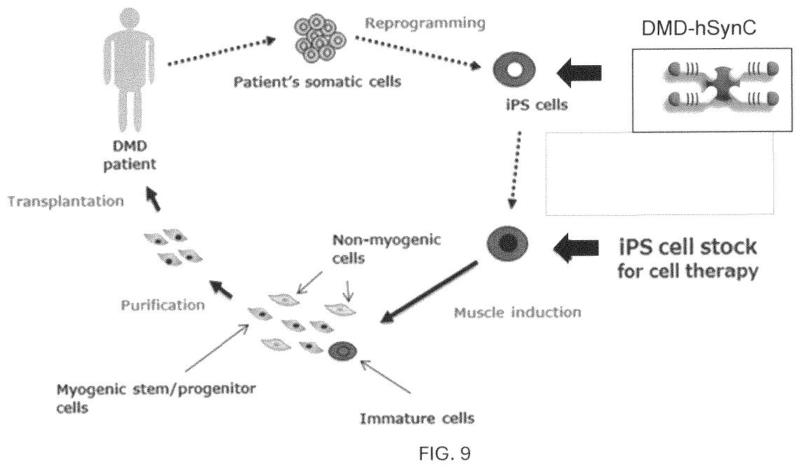

FIG. 9 graphically illustrates one method by which synthetic chromosomes expressing the full length DMD gene may be used to produce a cell therapy for treatment of Duchenne Muscular Dystrophy (as well as other cell therapies for other diseases). In this illustration, a patient's somatic cells are reprogrammed to become iPS cells, a DMD-encoding hSynC is introduced into the iPS cells, the cells containing the DMD-encoding hSynC are induced to become myogenic progenitor cells, and the myogenic progenitor cells are purified and transplanted into the patient.

The hSynC platform provides an adept bioengineering system enabling large genetic inputs onto a synthetic, chromosome-based vector without direct modification of the host genome. The hSynC proffers a novel cyto-reagent system amenable to designing complex genetic circuits for multitherapeutic biological delivery. Synthetic chromosomes, as engineerable modular platforms, interallied with advances in induced pluripotent stem cell production will greatly streamline the process and broaden the utility of precision medicine for pharmaceutical manufacturing geared towards the treatment of diseases such as muscular dystrophy.

The hSynC offers a versatile bioengineering tool that allows for the insertion of large genetic sequences into a synthetic chromosome, bypassing direct manipulation of the host genome.

The utility of the hSynC and similar synthetic chromosomes has been validated in genome engineering, protein production, animal modeling, and stem cell engineering applications. (Lindenbaum M, Perkins E, Csonka E, et al. Nucleic Acids Res. 2004, 32 (21): e172; Katona R L, Sinkó I, Holló G, et al. Cell. Mol. Life Sci. 2008, 65 (23): 3830-3838; Monteith D P, Leung J D, Borowski A H, et al. Methods Mol. Biol. 2004, 240:227-242).

The data presented herein have validated production of full-length dystrophin and isoform variants from the hSynC platform.

Importantly, addition of a genetic safety switch on the hSynC mitigates potential therapeutic side effects and makes the presently disclosed synthetic chromosome compositions exceptionally useful to precision medicine.

While various specific embodiments have been illustrated and described, it will be appreciated that various changes can be made without departing from the spirit and scope of the invention(s). Therefore, it is to be understood that the disclosure is not to be limited to the specific embodiments disclosed herein, as such are presented by way of example. Although specific terms are employed herein, they are used in a generic and descriptive sense only and not for purposes of limitation.

All literature and similar materials cited in this application, including, but not limited to, patents, patent applications, articles, books, treatises, internet web pages and other publications cited in the present disclosure, regardless of the format of such literature and similar materials, are expressly incorporated by reference in their entirety for any purpose to the same extent as if each were individually indicated to be incorporated by reference. In the event that one or more of the incorporated literature and similar materials differs from or contradicts the present disclosure, including, but not limited to defined terms, term usage, described techniques, or the like, the present disclosure controls.

What is claimed is:

1. An autonomously replicating, stably inherited, non-integrating, non-native mammalian synthetic chromosome (mSynC) comprising:
an rDNA-amplified centromere region;
at least two telomeres;
multiple copies of at least one type of unidirectional site-specific integration site;
at least one irreversibly integrated genetic cassette greater than 5 kbp in size, wherein the integrated cassette comprises at least one therapeutic gene;
a safety switch under tight expression control; and
a marker allowing for identification of mSynC-bearing cells.

2. The mSynC of claim 1, further comprising at least one additional element selected from:
a second therapeutic gene;
a lineage-specific cellular differentiation gene and/or regulatory sequence;
an enhancer of expression;
a sequence encoding a cell-surface protein;
a cellular growth factor; and
a cytokine.

3. The mSynC of claim 1, wherein the therapeutic gene is present in multiple copies on the mSynC.

4. The mSynC of claim 1, comprising at least one therapeutic gene involved in muscle function.

5. The mSynC of claim 4 wherein the therapeutic gene encodes a gene product that treats a muscular dystrophy.

6. The mSynC of claim 5, wherein the muscular dystrophy is selected from Duchenne Muscular Dystrophy (DMD), Limb-girdle Muscular Dystrophy (LGMD), myotonic dystrophy, Facioscapulohumeral Dystrophy (FSHD), Oculopharyngeal Muscular Dystrophy (CPMD), Oculopharyngeal Muscular Dystrophy (CPMD) and congenital muscular dystrophy.

7. The mSynC of claim 1, wherein at least one gene encodes the entire dystrophin protein.

8. The mSynC of claim 2, wherein the mSynC comprises at least the second therapeutic gene, and the second therapeutic gene is selected from:
another variant of the first therapeutic gene different from the first therapeutic gene, DP71ab, utrophin, dysferlin, acetylgalactosaminyltransferase, GALGT2, PAX7, nestin, calpain 3, alpha-, beta-, delta-, or gamma-sarcoglycan, desmin, and caveolin 3.

9. The mSynC of claim 8, comprising both the full-length DMD cDNA and the isoform Dp71ab.

10. The mSync of claim 8, wherein the mSynC further comprises at least one regulatory element that specifically regulates the second therapeutic gene.

11. The mSynC of claim 1, wherein multiple and different genes are present on the mSynC and, when inside host mammalian cells, express gene products that treat a complex disease having multiple causes.

12. The mSynC of claim 11, wherein the gene products are components of a multi-protein complex.

13. The mSynC of claim 11, wherein the gene products are expressed in the host cells at different levels.

14. A method of controlling expression of a therapeutic gene in a host cell employing the mSynC of claim 1.

15. A method of making a therapeutic cellular medicine by transferring the mSynC of claim 1 into a mammalian cell.

16. A method of cell-based therapy comprising:
transferring, ex vivo, the mSynC of claim 1 into a mammalian cell; and
administering the mSynC-carrying cells to a mammal in need of treatment.

17. The method of claim 15, wherein the mammalian cell is a progenitor cell, a satellite cell, a smooth muscle cell, a cardiac muscle cell, a skeletal muscle cell, a myoblast, a myotube, a syncytium, or a sarcomere.

18. A method of cell-based therapy comprising:
isolating autologous somatic cells from a patient,
reprogramming the patient-autologous cells to generate stem cells;
transferring, ex vivo, the mSynC of claim 1 into the stem cells to generate transgenic patient-autologous stem cells;
administering the transgenic patient-autologous stem cells carrying the mSynC to the patient.

19. The method of claim 18, further comprising reprogramming the patient-autologous cells to generate cells selected from: induced pluripotent stem cells (ipSCs), mesenchymal stem cells (MSCs), MSCs derived from umbilical cord (ucMSCs), myoblasts, mesoangioblasts (MABs), and human iPSC-derived MAB-like cells (HIDEMs).

20. The method of claim 18, wherein the autologous somatic cells are myoblasts, vessel cells, myotubes, muscle cells, adipose cells, bone marrow cells, cells from synovium, etc.

21. A cellular medicine for treating a disease, comprising mammalian cells carrying the mSynC of claim 1.

22. The cellular medicine of claim 21, wherein the disease is a Muscular Dystrophy.

23. The cellular medicine of claim 21, wherein the disease is DMD.

24. A host cell comprising the mSynC of claim 1.

25. The host cell comprising the mSynC of claim 24, wherein the host cell is a muscle cell.

26. The host cell comprising the mSynC of claim 24, wherein the host cell is a stem cell.

27. A cellular composition comprising the host cell of claim 24 and a pharmaceutically acceptable carrier.
